(19) United States
(12) Patent Application Publication
(10) Pub. No.: US 2025/0149115 A1
(43) Pub. Date: May 8, 2025

(54) CLINICAL GENETIC SCREENING ASSAY WITH RESCUE MINIMIZATION

(71) Applicant: Laboratory Corporation of America Holdings, Burlington, NC (US)

(72) Inventors: Eldar Yehuda Giladi, Arlington, MA (US); Qiandong Zeng, Belmont, MA (US); Angela Antoinette Kenyon, Worcester, MA (US); Stanley Ian Letovsky, Milton, MA (US)

(73) Assignee: Laboratory Corporation of America Holdings, Burlington, NC (US)

(21) Appl. No.: 18/936,882

(22) Filed: Nov. 4, 2024

Related U.S. Application Data
(60) Provisional application No. 63/597,272, filed on Nov. 8, 2023.

(51) Int. Cl. G16B 30/00; G06F 30/27; G16B 40/20

(52) U.S. Cl. CPC G16B 30/00; G06F 30/27; G16B 40/20

(57) ABSTRACT

The present disclosure relates to a clinical genetic screening assay that designates a subset of high-risk segments within a next generation sequencing (NGS) sample for confirmatory testing. Particularly, aspects are directed towards obtaining (i) regions of interest (ROIs) and associated variant data, (ii) population allele frequency information, and (iii) a sensitivity profile, performing NGS on a sample obtained from a subject to obtain NGS read data for the ROIs, performing a rescue minimization protocol to designate a subset of ROIs for Sanger resequencing, and performing Sanger resequencing on the subset of ROIs. The NGS read data and the Sanger resequencing data are used to generate accurate variant callings and/or diagnosis for the subject.

### CROSS-REFERENCE TO RELATED APPLICATION

The present application claims priority and benefit from U.S. Provisional Application No. 63/597,272, filed Nov. 8, 2023, the entire contents of which are incorporated herein by reference for all purposes.

### FIELD

The present disclosure relates to a clinical genetic screening assay, in particular, to a clinical genetic screening assay that will reduce the total false negative variant call risk of the sample by designating the highest risk segments within a next generation sequencing sample for confirmatory testing of variant calls to minimize the time and resources used in sample reprocessing.

### BACKGROUND

Rapid advancement in sequencing technologies has revolutionized routine diagnostics for detecting genetic mutations in clinical laboratories around the world. The primary sequencing technologies in use in the clinical setting include Sanger sequencing and Next Generation Sequencing (NGS) methods. Sanger sequencing is a first-generation DNA sequencing method that has long been considered the gold standard for the accurate detection of single nucleotide variants (SNVs). First generation sequencing techniques, like Sanger, utilize a chain-termination method wherein specialized DNA bases (dideoxynucleotides or ddNTPs) are randomly incorporated into a growing DNA chain of nucleotides (A, C, G, T) generating different length DNA fragments. Fragments are size separated by capillary electrophoresis and a laser is used to excite the unique fluorescence signal associated with each ddNTP. As the fluorescence signal is recorded, a chromatograph is generated, showing which base is present at a given location of the target region being sequenced. In the clinical setting, Sanger provides flexibility for testing single or small batch samples (no more than a 10 gene panel) for prenatal, carrier, and genetic testing and can provide results in a relatively short period of time. However, Sanger is limited to short DNA sequences, approximately 300-1,000 bases, and can be more expensive compared to newer generation sequencing methods. Further, many genetic conditions can have over 40 disease causing genes, making screening by Sanger infeasible.

NGS has largely replaced Sanger sequencing in routine clinical testing, due to its superior capabilities in throughput, coverage, and cost-effectiveness. NGS can sequence millions to billions of bases of DNA fragments simultaneously, instead of just a few hundred by Sanger, enabling comprehensive genomic analyses that detect a wide range of genetic variations, including rare mutations, with high sensitivity and accuracy. For example, NGS has proven to perform nearly as accurately as Sanger for variant detection with concordance rates of >99% being reported for SNVs and indels in high-complexity regions. This high-throughput approach reduces the cost per base of sequencing, making it more economical for large-scale projects. Additionally, NGS provides faster turnaround times for extensive genomic data, which is beneficial for applications like personalized medicine and complex disease diagnostics. In contrast, Sanger sequencing, while reliable for small-scale, targeted gene sequencing, is slower, less comprehensive, and becomes prohibitively expensive for larger genomic studies. These advancements have greatly improved the flexibility of genetic diagnostics, providing highly sensitive and accurate high-throughput platforms for genome-scale testing.

Targeted genomic sequencing (TGS), whole genome sequencing (WGS), and whole exome sequencing (WES) are three distinct sequencing approaches used in the analysis of genetic material, each with its own unique applications and benefits. TGS focuses on a panel of genes or targets known to contain DNA alterations with strong associations to the pathogenesis of disease and/or clinical relevance. DNA alterations typically include single nucleotide variants (SNVs), deletions and/or insertions (indels), inversions, translocations/fusions, and copy number variations (CNV). Because only specific regions of interests from the genome are interrogated in TGS, a much greater sequencing depth is achieved (number of times a given nucleotide is sequenced), and highly accurate variant calls are obtained at a significantly reduced cost and data burden compared to more global NGS methods such as WGS and WES. Moreover, TGS can identify low frequency variants in targeted regions with high confidence and is thus suitable for profiling low-quality and fragmented clinical DNA samples (e.g., as seen in cell-free DNA). This approach is often employed in clinical settings where specific genetic markers are being investigated, such as in the diagnosis of certain cancers or inherited genetic disorders.

WGS, on the other hand, involves sequencing the entire genome, providing a comprehensive overview of all genetic material, including coding and non-coding regions (e.g., covering all or substantially all the 3 billion DNA base pairs that make up an entire human genome). WGS offers an unbiased approach to genetic analysis, capturing a wide array of genetic variations, including single nucleotide variants, insertions, deletions, copy number variations, and structural variants. This method is invaluable for research and clinical diagnostics when a holistic view of the genome is required, for instance, in complex diseases with multifactorial genetic contributions such as cancer diagnostics. Whole exome sequencing falls somewhere in between TGS and WGS. WES focuses exclusively on the exonic regions of the genome, which constitute about 1-2% of the genome but harbor approximately 85% of known disease-causing mutations. WES provides a more cost-effective solution than WGS while still covering a significant portion of clinically relevant genetic information, making it a popular choice for diagnosing certain diseases (e.g., Mendelian disorders) and uncovering novel genetic mutations linked to diseases.

### SUMMARY

In various embodiments, a computer-implemented method is provided that comprises: performing a next generation sequencing (NGS) on a patient sample, wherein the NGS generates NGS read data for one or more regions of interest, wherein the one or more regions of interest comprise segments, the segments comprise one or more positions previously reported to have variants, and the variants comprise alterations to a DNA sequence not found in a reference sequence; inputting the NGS read data into a rescue minimization protocol, wherein the rescue minimization protocol comprises a preprocessing process comprising generating input data based on the NGS read data for the one or more regions of interest; and a rescue minimization process comprising: accessing the input data, calculating an expected false negative (EFN) value for each of the segments; sorting the segments based on their EFN values, in an iterative subprocess, calculating the EFN value for the patient sample based on a sum over all the EFN values across the segments, comparing the sum of EFN values for the patient sample to a threshold and when the sum of EFN values for the patient sample is greater than the threshold, removing one or more of the segments from the iterative subprocess that contribute the most to the EFN value for the patient sample based on segment's EFN values, repeating the iterative subprocess until the sum of all the remaining EFN values for the patient sample is less than or equal to the threshold, and outputting the one or more segments removed in the iterative subprocess and designating them for rescue; performing Sanger sequencing on the one or more segments designated for rescue, wherein the Sanger sequencing generates confirmatory read data for the one or more regions of interest; and outputting a result of the clinical genetic screening assay using a combination of the variants from the NGS read data and the confirmatory read data for the one or more regions of interest.

In some embodiments, the computer-implemented method further comprises: selecting a genetic panel configured to detect the alterations in the DNA sequence in the one or more regions of interest, wherein the genetic panel comprises probes for the one or more regions of interest, and wherein the NGS is targeted NGS performed using the probes.

In some embodiments, the targeted NGS read data comprise high-risk segments, wherein the high-risk segments are the segments at risk of having a false negative variant call and are designated for rescue by Sanger confirmatory sequencing.

In some embodiments, the variants further comprise pathogenic or likely pathogenic variants, or variants highly predicted to affect the sequence of its gene, transcript, protein, or any combination thereof.

In some embodiments, the preprocessing process further comprises accessing one or more datasets, classifying variants into groups, generating variant sensitivity profiles, and saving all data files into data structures, which can be accessed by the rescue minimization process.

In some embodiments, the one or more datasets include (i) a dataset of pathogenic and likely pathogenic variants and (ii) a dataset of variants highly predicted to affect the sequence of its gene, transcript, protein, or any combination thereof.

In some embodiments, the dataset of pathogenic and likely pathogenic variants and the dataset of variants highly predicted to affect the sequence of its gene, transcript, protein, or any combination thereof are overlapped with the NGS read data and generate a final dataset that comprises a list of segments that contain one or more positions previously reported to have either a pathogenic or likely pathogenic variant, a variants highly predicted to affect the sequence of its gene, transcript, protein, or any combination thereof, or both.

In some embodiments, classifying variants into groups comprises: accessing sequencing read data for a benchmark variant dataset, wherein the benchmark variant dataset comprises high confidence variants; classifying the high confidence variants based on a mappability score and when the mappability score is below 0.5 grouping the high confidence variants into a low mappability region variant group; and classifying the high confidence variants based on zygosity, variant type, and variant length and grouping the variants into either a heterozygous single nucleotide variants group, a short heterozygous indels group, or a long heterozygous indels group.

In some embodiments, generating variant sensitivity profiles comprises: accessing the variant groups, wherein the variant group accessed is the heterozygous single nucleotide variants group; titrating the read coverage, for the heterozygous single nucleotide variants group, to range from 1-90% of the original read coverage; simulating variant detection, for the titrated reads, using NGS analysis and variant calling; reporting out whether the heterozygous single nucleotide variants are detected; and building a heterozygous single nucleotide variant sensitivity profile of the heterozygous single nucleotide variants to determine the probability of detecting a heterozygous single nucleotide variant given a specific read coverage at the position of the heterozygous single nucleotide variant.

In some embodiments, the variant group accessed further includes the short heterozygous indels group, the long heterozygous indels group, and the low mappability region variant group and a variant sensitivity profile is built for the short heterozygous indels group, the long heterozygous indels group, and the low mappability region variant group.

In some embodiments, the rescue minimization process further comprises: calculating EFN values for the one or more positions previously reported to have variants by subtracting the probability of detecting a variant given a specific read coverage at that position and the variant type from 1 and then multiplying by the population frequency of the variant, wherein the EFN values for the one or more positions previously reported to have variants are based on the conditional probability that a variant is not detected at a specific location given the read coverage at that position and the variant type; and calculating the EFN value for a segment based on a sum over all the EFN values across the one or more positions previously reported to have variants within the segment.

In some embodiments, the result of the clinical genetic screening assay comprises the variants detected and their clinical significance, the type of variant and which base or bases are altered from the reference sequence, and which variants were reprocessed by Sanger sequencing.

In various embodiments, a computer-implemented method is provided for performing a clinical genetic screening assay comprising: obtaining a plurality of regions of interest (ROIs) comprising a set of variants, wherein the set of variants comprises pathogenic variants, likely pathogenic variants, and/or computationally predicted high impact variants; obtaining population allele frequency information for the set of variants, wherein the population frequency information comprises a population allele frequency for each variant of the set of variants; performing next generation sequencing (NGS) on a patient sample to generate NGS read data for the plurality of ROIs; determining a read coverage for each location of each ROI of the plurality of ROIs based on the NGS read data; obtaining a sensitivity profile, wherein the sensitivity profile provides a probability that a variant is detected based on a read coverage and a variant type of the variant; performing a rescue minimization process comprising: determining an expected false negative (EFN) value for each ROI of the plurality of ROIs based on the sensitivity profile and a population allele frequency of one or more variants in the ROI, determining a sample EFN value for the patient sample based on the EFN values of the plurality of ROIs, determining if the sample EFN value is greater than a predetermined threshold, and when the sample EFN value is greater than the predetermined threshold, sorting the plurality of ROIs based on the EFN values; and rescuing a number of ROIs from the plurality of ROIs based on the sorting, wherein a sum of the EFN values of remaining ROIs of the plurality of ROIs is less than or equal to the predetermined threshold; performing Sanger sequencing on the rescued ROIs, wherein the Sanger sequencing generates confirmatory read data for the one or more ROIs; and outputting a result of the clinical genetic screening assay based on the NGS read data and the confirmatory read data.

In some embodiments, the NGS is targeted NGS targeting the plurality of ROIs.

In some embodiments, the computer-implemented method further comprises excluding a subset of ROIs from the plurality of ROIs from the rescue minimization process, wherein each ROIs of the subset of ROIs has a read coverage below a predetermined minimum threshold, wherein the Sanger sequencing is further performed on the subset of ROIs.

In some embodiments, the predetermined minimum threshold is 30×, 20×, 10× or less than 10×.

In some embodiments, the high impact variants are determined by a computational mutation impact analysis method.

In some embodiments, the computer-implemented method further comprises determining the sensitivity profile by modeling variant data obtained from one or more databases based on logistic regression or using a piecewise model.

In some embodiments, when the sensitivity profile is determined using the piecewise model, and the piecewise model is a piecewise logistic regressing model.

In some embodiments, the piecewise logistic regressing model is:

$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$

$$wherein :$$

$$\frac{{e}^{a + b \times T}}{1 + {e}^{a + b \times T}} = \frac{{e}^{c + d \times T}}{1 + {e}^{c + d \times T}}$$

wherein a, b, c, d, and T are predetermined parameters.

In some embodiments, the variant types comprise homozygous variants, heterozygous single nucleotide variants (SNVs), heterozygous short deletions and/or insertions (indels), heterozygous long indels, and/or low mappability variants.

In some embodiments, the variant data is obtained by titrating read coverage and simulating variant detection for the titrated reads using NGS analysis and variant calling.

In some embodiments, the computer-implemented method further comprises determining a population allele frequency for each variant of the set of variants based on variant data obtained from one or more databases comprising clinically relevant variants.

In some embodiments, the population allele frequencies are determined based on variant data obtained from one or more databases.

In some embodiments, a default allele frequency is determined to be the population allele frequency for a variant that is not in the one or more databases, wherein the default allele frequency is determined by extrapolating a power law using the variant data obtained from the one or more databases.

In some embodiments, the predetermined threshold is 1/5000, 1/10000, 1/15000, or 1/20000.

In some embodiments, the computer-implemented method further comprises classifying high confidence variants into groups comprises: accessing sequencing read data for a benchmark variant dataset, wherein the benchmark variant dataset comprises the high confidence variants; determining a mappability score for each of the high confidence variants; grouping a first subset of the high confidence variants into a low mappability group, wherein each variant in the first subset has a mappability score of below a predetermined value; and grouping remaining high confidence variants into a heterozygous single nucleotide variants group, a short heterozygous indels group, or a long heterozygous indels group based on each variant's zygosity, variant type, and variant length.

In some embodiments, each variant in the short heterozygous indels group has 1-5 affected base pairs, and each variant in the long heterozygous indels group has greater than 5 affected base pairs.

In some embodiments, a computer-program product is provided that is tangibly embodied in a non-transitory machine-readable medium and that includes instructions configured to cause one or more data processors to perform part or all of one or more methods disclosed herein.

In some embodiments, a system is provided that includes one or more data processors and a non-transitory computer readable medium containing instructions which, when executed on the one or more data processors, cause the one or more data processors to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the techniques claimed. Thus, it should be understood that although the present techniques have been specifically disclosed by embodiments and optional features, modification and variation of the concepts herein disclosed may be resorted to by those skilled in the art, and that such modifications and variations are considered to be within the scope of this application as defined by the appended claims.

### BRIEF DESCRIPTION OF THE DRAWINGS

The present disclosure will be better understood in view of the following non-limiting figures, in which:

FIG. 1 shows a computing environment in accordance with various embodiments;

FIG. 2 shows an illustration of a clinical genetic screening assay in accordance with various embodiments;

FIG. 3 shows an illustration of a targeted NGS sample broken down into components in accordance with various embodiments;

FIG. 4 shows a graph displaying the power law for allele frequency counts in Genome Aggregation Database (gnomAD) in accordance with various embodiments;

FIG. 5 shows a flowchart illustrating a process for identifying segments from a sequencing sample that require rescue resequencing in accordance with various embodiments;

FIG. 6 shows a flowchart illustrating the rescue minimization pipeline in accordance with various embodiments;

FIGS. 7A and 7B illustrate relationship between read coverage and probability of detecting the variant, and different smoothing models to simulate the relationship, in accordance with various embodiments;

FIGS. 8A-8B illustrate tradeoff relationship between the number of rescues per sample and reprocessing in accordance with various embodiments; and

FIG. 9 illustrates the cumulative distribution (CDF) of the resulting EFN values for all the samples in the rescue minimization approach with F= 1/5000, and the coverage-based approach with minimum coverage of 15×, in accordance with various embodiments.

### TERMS

As used herein, the singular forms “a,” “an,” and “the” include plural references unless the context clearly dictates otherwise. For example, references to “the method” include one or more methods, and/or steps of the type described herein, which will become apparent to those persons skilled in the art upon reading this disclosure and so forth. Additionally, the term “a nucleic acid” includes a plurality of nucleic acids, including mixtures thereof.

As used herein, the terms “about” and “approximately” are used interchangeably and mean within an acceptable error range for the particular value as determined by one of ordinary skill in the art, and thus depend in part on how the value is measured or determined, e.g., the limitations of the measurement system. For example, the term “substantially,” “approximately,” or “about” may be substituted with “within [a percentage] of” what is specified, where the percentage includes 0.1, 1, 5, and 10 percent. Where particular values are described in the application and claims, unless otherwise stated, the term “about” means within an acceptable error range for the particular value.

As used herein, the terms “aligned,” “alignment,” and “aligning” refer to two or more nucleic acid sequences that can be identified as a match (e.g., 100% identity) or partial match (e.g., less than 100% identity).

As used herein, the term “allele” refers to any alternative forms of a gene at a particular locus. There may be one or more alternative forms, all of which may relate to one trait or characteristic at the specific locus. In a diploid cell of an organism, alleles of a given gene can be located at a specific location, or locus (loci plural) on a chromosome. The genetic sequences that differ between different alleles at each locus are termed “variants,” “polymorphisms,” or “mutations.” The term “single nucleotide polymorphisms” (SNPs) can be used interchangeably with “single nucleotide variants” (SNVs).

As used herein, the term “allele frequency” or “allelic frequency” refers to the relative frequency of an allele (e.g., variant of a gene) in a sample, e.g., expressed as a fraction or percentage. In some cases, allelic frequency may refer to the relative frequency of an allele (e.g., variant of a gene) in a sample, such as a cell-free nucleic acid (CFNA) sample. The allelic frequency of a mutant allele may refer to the frequency of the mutant allele relative to the wild-type allele in a sample. For example, if a sample includes 100 copies of a gene, five of which are a mutant allele and 95 of which are the wild-type allele, an allelic frequency of the mutant allele is about 5/100 or about 5%. A sample having no copies of a mutant allele (e.g., about 0% allelic frequency) may be used as a control sample or a reference sample, for example, as a negative control. A negative control may be a sample in which no mutant allele is expected to be detected. A sample including a mutant allele at about 50% allelic frequency may, for example, be representative of a germline heterozygous mutation.

As used herein, when an action is “based on” something, this means the action is based at least in part on at least a part of the something.

As used herein, the term “cell-free nucleic acid” or “CFNA” refers to extracellular nucleic acids, as well as circulating free nucleic acid. As such, the terms “extracellular nucleic acid,” “cell-free nucleic acid” and “circulating free nucleic acid” are used interchangeably. Extracellular nucleic acids can be found in biological sources such as blood, urine, and stool. CFNA may refer to cell-free DNA (cfDNA), circulating free DNA (cfDNA), cell-free RNA (cfRNA), or circulating free RNA (cfRNA).

As used herein, the term “copy number alteration,” “copy number variation,” or “CNV” refers to a class or type of genetic variation, genetic alteration or chromosomal aberration. In certain instances, “copy number alteration,” “copy number variation,” or “CNV” may be used to describe a somatic alteration whereby the genome in a subset of cells in a subject contains the alteration (such as, for example, in tumor or cancer cells). In certain instances, “copy number alteration,” “copy number variation,” or “CNV” may be used to describe a variation inherited from one or both parents (such as, for example, a copy number variation in a fetus). “Copy number alteration,” “copy number variation,” or “CNV” can be a deletion (e.g., microdeletion), duplication (e.g., a microduplication) or insertion (e.g., a microinsertion). In some instances, the prefix “micro” refers to a region of nucleic acid less than 7 Mb in length, less than 5 Mb in length, or less than 1 Mb in length. A “copy number alteration,” “copy number variation,” or “CNV” can include one or more deletions (e.g., microdeletion), duplications and/or insertions (e.g., a microduplication, microinsertion) of a part of a chromosome or the whole chromosome. In certain embodiments, a duplication comprises an insertion. In certain embodiments, an insertion is a duplication.

As used herein, the term “genetic screening,” “genetic testing,” or “genetic screening test” refers to a process of testing individuals or populations for specific genetic traits, mutations, or abnormalities that may indicate a predisposition to certain diseases, conditions, or inherited disorders, including but not limited to the followings: prenatal genetic screening tests (e.g., non-invasive prenatal testing (NIPT) or non-invasive prenatal screening (NIPS), first trimester screening, second trimester screening (quad screen), carrier screening, amniocentesis, and chorionic villus sampling (CVS)); newborn screening tests (e.g., the heel prick test (Guthrie test)); cancer genetic screening (e.g., BRCA1 and BRCA2 testing, Lynch syndrome screening, and FAP (familial adenomatous polyposis) testing); cardiovascular genetic screening (e.g., familial hypercholesterolemia testing and hypertrophic cardiomyopathy testing); neurological genetic screening (e.g., Huntington's disease testing and Alzheimer's disease genetic testing); metabolic and other genetic disorders screening (e.g., cystic fibrosis testing, thalassemia and sickle cell disease testing, and hemochromatosis testing); pharmacogenomic testing includes (e.g., cytochrome P450 testing); ancestry and health-related genetic screening; rare disease screening (e.g., exome sequencing and whole genome sequencing); genetic screening for specific populations; carrier screening; and prenatal and preconception screening (e.g., expanded carrier screening). In some instances, “genetic screening” refers to “carrier screening.”

As used herein, the term “likely” refers to a probability range of about 80%-99% when describing the significance of an event. In some embodiments, “likely” is 95%-98%. For example, a “likely benign” variant has a 95%-98% chance of being benign, and a “likely pathogenic” variant has a 95%-98% chance of being pathogenic. Different ranges may be used for different events.

As used herein, the term “mutant” or “variant,” when made in reference to an allele or sequence, generally refers to an allele or sequence that does not encode the phenotype most common in a particular natural population. The terms “mutant allele” and “variant allele” can be used interchangeably. In some cases, a mutant allele can refer to an allele present at a lower frequency in a population relative to the wild-type allele. In some cases, a mutant allele or sequence can refer to an allele or sequence mutated from a wild-type sequence to a mutated sequence that presents a phenotype associated with a disease state and/or drug resistant state. Mutant alleles and sequences may be different from wild-type alleles and sequences by only one base but can be different up to several bases or more. The term “mutant” when made in reference to a gene generally refers to one or more sequence mutations in a gene, including a point mutation, a SNP, an insertion, a deletion, a substitution, a transposition, a translocation, a copy number variation, or another genetic mutation, alteration, or sequence variation. In some instances, the term “mutation” is used interchangeably with “alteration” or “variant.”

As used herein, the term “nucleic acid” or “nucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have comparable properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

As used herein, the term “portion,” “genomic section,” “bin,” “partition,” “portion of a reference genome,” “portion of a chromosome” or “genomic portion” refers to a product by partitioning of a genome according to one or more features. Non-limiting examples of certain partitioning features include length (e.g., fixed length, non-fixed length) and other structural features. Genomic portions sometimes include one or more of the following features: fixed length, non-fixed length, random length, non-random length, equal length, unequal length (e.g., at least two of the genomic portions are of unequal length), do not overlap (e.g., the 3′ ends of the genomic portions sometimes abut the 5′ ends of adjacent genomic portions), overlap (e.g., at least two of the genomic portions overlap), contiguous, consecutive, not contiguous, and not consecutive. Genomic portions sometimes are about 1 to about 1,000 kilobases in length (e.g., about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900 kilobases in length), about 5 to about 500 kilobases in length, about 10 to about 100 kilobases in length, or about 40 to about 60 kilobases in length.

As used herein, the term “read” or “sequence read” is a short nucleotide sequence produced by any sequencing process, including NGS, described herein or known in the art.

As used herein, the term “sample,” “biological sample,” “patient sample,” “tissue,” and “tissue sample” refer to any sample including a biomolecule (such as a protein, a peptide, a nucleic acid, a lipid, a carbohydrate, or a combination thereof) that is obtained from any organism including viruses, and the terms may be used interchangeably. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the terms “standard” or “reference,” as used herein, generally refer to a substance which is prepared to certain pre-defined criteria and can be used to assess certain aspects of, for example, an assay. Standards or references preferably yield reproducible, consistent, and reliable results. These aspects may include performance metrics, examples of which include, but are not limited to, accuracy, specificity, sensitivity, linearity, reproducibility, limit of detection and/or limit of quantitation. Standards or references may be used for assay development, assay validation, and/or assay optimization. Standards may be used to evaluate quantitative and qualitative aspects of an assay. In some instances, applications may include monitoring, comparing and/or otherwise assessing a QC sample/control, an assay control (product), a filler sample, a training sample, and/or lot-to-lot performance for a given assay.

As used herein, the term “segment” or “genomic segment” refers to one or more genomic portions, and often includes one or more consecutive portions (e.g., about 2 to about 100 such portions (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90 such portions)). A segment or genomic segment is a part of the target chromosome, gene, exon, intron or other region of interest. In some instances, a segment can include non-consecutive portions. The term “segment” may be used interchangeably with “region of interest,” “ROI,” or “target region.”

As used herein, the term “sequence variant” refers to any variation in sequence relative to one or more reference sequences. A sequence variant may occur with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the sequence variant occurs with a low frequency in the population (also referred to as a “rare” sequence variant). For example, in non-tissue samples, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some non-tissue sample cases, the sequence variant occurs with a frequency of about or less than about 0.1%. In tissue, the sequence variant may occur with a frequency of about or less than about 100%, 90%, 80%, 70%, 60%, 50%, 40%, 30%, 20%, 10%, 5%, or lower. A sequence variant can be any sequence that varies from a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a sequence variant includes two or more nucleotide differences, the nucleotides that are different may be contiguous with one another, or discontinuous. Non-limiting examples of types of sequence variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of sequence variants include those that occur within short tandem repeats (STR) and simple sequence repeats (SSR), or those occurring due to amplified fragment length polymorphisms (AFLP) or differences in epigenetic marks that can be detected (e.g., methylation differences). In some instances, a sequence variant can refer to a chromosome rearrangement, including but not limited to a translocation or fusion gene, or rearrangement of multiple genes resulting from, for example, chromothripsis. As used herein, the term “reference genome” can refer to any particular known, sequenced or characterized genome, whether partial or complete, of any organism or virus which may be used to reference identified sequences from a subject.

As used herein, the term “wild type” when made in reference to an allele or sequence, refers to the allele or sequence that encodes the phenotype most common in a particular natural population. In some cases, a wild-type allele can refer to an allele present at highest frequency in the population. In some cases, a wild-type allele or sequence refers to an allele or sequence associated with a normal state relative to an abnormal state, for example a disease state.

Unless defined otherwise, all technical and scientific terms used herein have the same meaning as commonly understood by one of ordinary skill in the art to which this disclosure belongs. Although any methods and materials similar to or equivalent to those described herein can be used in the practice or testing of the application, the preferred methods and materials are now described.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

NGS has become very popular in clinical care and research due to its massively parallel sequencing abilities; however, Sanger sequencing remains the current standard of care for validating variants detected by NGS. This is despite several studies reporting that NGS is just as accurate, when appropriate quality thresholds are met, with concordance rates of >99% being reported for SNVs and indels in high-complexity regions. As a result, Sanger is taking on a new role where it is mostly being used to confirm variant calls in regions where NGS is unable to achieve sufficient depth coverage, regions with homology to other regions, regions with low complexity, repeat expansions, and methylation, or before variants are clinically reported.

The continued advancement in sequencing technologies has opened the door for the discovery and detection of even more disease-causing variants, allowing clinicians to better serve their patients. However, the increased demand for genetic screenings has exponentially increased the number of samples being submitted to the laboratories for testing. More specifically, in terms of scalability and throughput. NGS platforms are designed for high-throughput sequencing, enabling the analysis of large volumes of data efficiently. However, scaling up NGS operations to meet increased demand requires substantial investment in additional equipment, software, and skilled personnel. This expansion can be both costly and time-consuming. Furthermore, the verification of NGS results using Sanger sequencing, a more labor-intensive and lower-throughput method, can create bottlenecks in the workflow. The nature of Sanger sequencing verification processes can slow down the overall turnaround time for delivering conclusive results, thereby impacting the timely diagnosis and treatment of patients in clinical settings.

Additionally, the challenges extend to cost and resource allocation, data management, and quality control. While NGS is cost-effective for large-scale sequencing projects, the initial setup and ongoing maintenance of NGS infrastructure require significant financial outlay. The cost of reagents and consumables for Sanger sequencing verification adds to the financial burden, especially when verifying numerous NGS findings. Managing the vast amount of data generated by NGS necessitates robust bioinformatics pipelines and data storage solutions, which require specialized expertise and technology. Integrating and ensuring consistency between NGS and Sanger sequencing data can be complex and time-consuming. Moreover, maintaining high-quality standards for both NGS and Sanger sequencing involves rigorous quality control measures and adherence to regulatory standards, which can further complicate the workflow and increase the demand for meticulous oversight and standardization. In an effort to mitigate these challenges and continue to provide the highest quality of care to patients, approaches aimed at minimizing NGS verification by Sanger sequencing are being explored.

One such example has been the incorporation of machine learning technology to predict if a variant is eligible for bypass of Sanger resequencing confirmation based on a set of quality features associated with different variant types, as described in U.S. Provisional Application No. 63/597,231 the entire contents of which are incorporated herein by reference for all purposes. This method reduces the total number of variants previously requiring Sanger confirmation down to 15% or less by only resequencing those regions containing variant calls not designated for bypass of Sanger sequencing. The clinical genetic screening assay and techniques presented herein, compliment and further improve upon the techniques described in U.S. Provisional Application No. 63/597,231 by designating only a portion of segments of a sample that contain the challenging or clinically relevant variants for Sanger confirmation instead of the entire set of low-coverage segments of the sample, for Sanger reprocessing. The disclosed techniques address the challenges and limitations associated with the increasing need in clinical laboratories to provide accurate, timely, and cost-effective genetic screening services for their patients.

More specifically, by designating only a portion of segments of a sample that contain the challenging or clinically relevant variants for Sanger confirmation instead of the entire set of low-coverage segments of the sample, the challenges associated with the increased demand for NGS screening assays can be significantly alleviated. By prioritizing the verification of only the most clinically significant or uncertain NGS results, laboratories can reduce the bottlenecks and delays caused by the time-consuming Sanger sequencing process. This targeted approach allows for more efficient use of resources, enabling laboratories to handle higher volumes of NGS data without compromising the overall turnaround time for delivering results. Consequently, this can lead to faster and more timely diagnoses and treatment decisions for patients. Additionally, reducing the reliance on Sanger sequencing for verification can lower the costs associated with reagents, consumables, and labor required for the sequencing process. This cost-saving measure can allow laboratories to allocate more resources towards enhancing NGS infrastructure, investing in advanced bioinformatics tools, and training personnel. Moreover, focusing Sanger verification on critical cases can help maintain high-quality standards and regulatory compliance more effectively, as the quality assurance processes can be streamlined and concentrated on fewer, but more impactful, verifications. Overall, this strategy balances the need for thorough validation with the practicalities of resource management and efficiency in clinical genetic testing.

As described herein, the present clinical genetic screening assay and techniques comprise performing NGS on a patient sample and performing a rescue minimization process to segments based on their NGS read data, population allele frequency information, and a sensitivity profile. The population allele frequency information can be used to estimate how likely a pathogenic or likely pathogenic variant might be present in a particular sample. The sensitivity profile provides a probability of a variant to be detected based on a read coverage and its variant type. By considering the population allele frequency information and the sensitivity profile, the rescue minimization process can accurately assess the risk of a false negative variant call being made in the segments, and evaluate if the false negative calling rate for the patient sample is acceptable. Segments with highest expected false negative values can be removed to keep the false negative calling rate for the patient sample below a predetermined threshold, and they are designated for confirmatory sequencing by Sanger (i.e., are “rescued”). By prioritizing the subset of “high-risk” segments contributing the most to false negative variant calls, the amount of time and resources previously expended on whole sample processing for all low-coverage segments in a sample is reduced to a fraction of the original cost. Further, this novel approach for sample reprocessing and data analysis provides a solution to the rising demand of clinical laboratories to provide accurate, timely and cost-effective services to their patients.

In various embodiments, a computer-implemented method is provided for performing a clinical genetic screening assay comprising: obtaining a plurality of regions of interest (ROIs) comprising a set of variants, wherein the set of variants comprises pathogenic variants, likely pathogenic variants, and/or computationally predicted high impact variants; obtaining population allele frequency information for the set of variants, wherein the population allele frequency information comprises a population allele frequency for each variant of the set of variants; performing next generation sequencing on a patient sample to generate NGS read data for the plurality of ROIs; determining a read coverage for each location of each ROI of the plurality of ROIs based on the NGS read data; obtaining a sensitivity profile, wherein the sensitivity profile provides a probability that a variant is detected based on a read coverage and a variant type of the variant; performing a rescue minimization process comprising: determining an expected false negative (EFN) value for each ROI of the plurality of ROIs based on the sensitivity profile and a population allele frequency of one or more variants in the ROI, determining a sample EFN value for the patient sample based on the EFN values of the plurality of ROIs, determining if the sample EFN value is greater than a predetermined threshold, and when the sample EFN value is greater than the predetermined threshold, sorting the plurality of ROIs based on the EFN values; and rescuing a number of ROIs from the plurality of ROIs based on the sorting, wherein a sum of the EFN values of remaining ROIs of the plurality of ROIs is less than or equal to the predetermined threshold; performing Sanger sequencing on the rescued ROIs, wherein the Sanger sequencing generates confirmatory read data for the one or more ROIs; and outputting a result of the clinical genetic screening assay based on the NGS read data and the confirmatory read data.

### II. Computing Environment

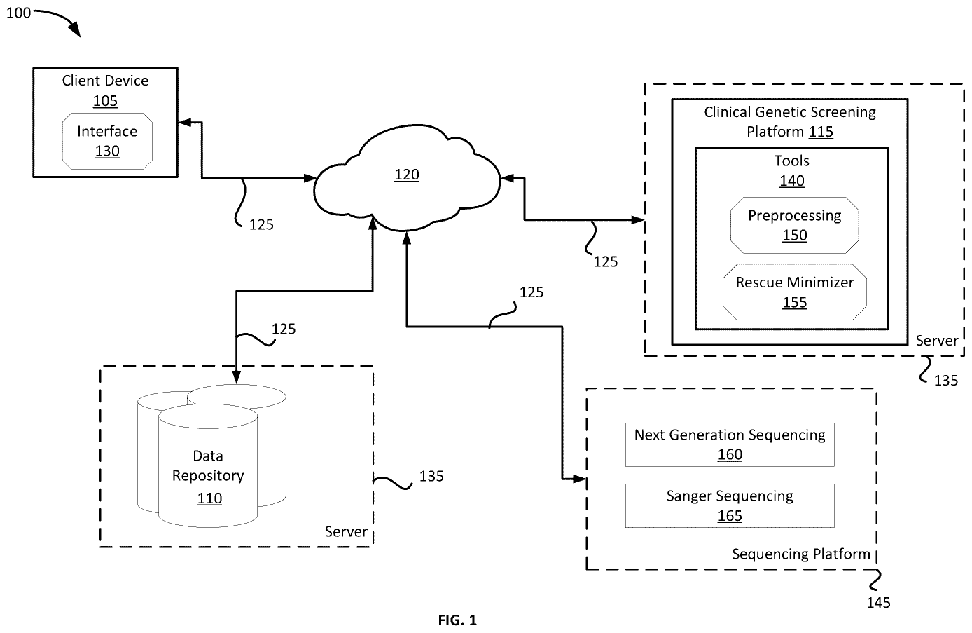

FIG. 1 shows a computing environment 100 in accordance with various embodiments. Computing environment 100 includes a client device 105, a server 135, a sequencing platform 145, and a network 120 connecting to components of the computing environment 100. Although FIG. 1 illustrates a particular arrangement of the client device 105, the server 135, the sequencing platform 145, and the network 120, this disclosure contemplates any suitable arrangement of these components and additional components. As an example, and not by way of limitation, two or more client devices 105, the server 135, and the sequencing platform 145 may be connected to each other directly, bypassing the network 120. As another example, two or more client devices 105, the server 135, and the sequencing platform 145 may be physically or logically co-located with each other in whole or in part. Moreover, although FIG. 1 illustrates a particular number of the components, this disclosure contemplates any suitable number of components (e.g., client devices 105, servers 135, sequencing platforms 145, and networks 120). As an example, and not by way of limitation, computing environment 100 may include multiple client devices 105, multiple servers 135, multiple sequencing platforms 145, and multiple networks 120.

This disclosure contemplates any type of network 120 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 120 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 125 may connect a client device 105, a server 135 or a unit thereof (e.g., a data repository 110, a clinical genetic screening platform 115), or a sequencing platform 145 or a unit thereof (e.g., a NGS unit 160, or a Sanger sequencing unit 165) to a network 120 or to each other. This disclosure contemplates any suitable links 125. In particular embodiments, one or more links 125 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 125 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 125, or a combination of two or more such links 125. Links 125 need not necessarily be the same throughout the computing environment 100. One or more first links 125 may differ in one or more respects from one or more second links 125.

A client device 105 is an electronic device including hardware, software, or embedded logic components or a combination of two or more such components and capable of interacting with the server 135 or a unit thereof (e.g., the data repository 110, the clinical genetic screening platform 115) and the sequencing platform 145 or a unit thereof (e.g., the NGS unit 160, the Sanger sequencing unit 165), optionally via the network 120. The client device 105 may include various types of computing systems such as portable handheld devices, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device 105 may be capable of executing various different applications such as various Internet-related apps, communication applications (e.g., E-mail applications, short message service (SMS) applications) and may use various communication protocols. This disclosure contemplates any suitable client device 105 configured to generate and output product target discovery content to a user. For example, users may use client device 105 to execute one or more applications, which may generate one or more discovery or storage requests that may then be serviced in accordance with the teachings of this disclosure. The client device 105 may provide an interface 130 (e.g., a graphical user interface) that enables a user of the client device 105 to interact with the client device 105. The client device 105 may also output information to the user via this interface 130 (e.g., displaying a report). Although FIG. 1 depicts only one client device 105, any number of client devices 105 may be supported.

The client device 105 is capable of inputting data, generating data, and receiving data. For example, a user of the client device 105 may send out a request to perform a clinical genetic screening assay using the interface 130. The request may be sent out through the network 120 to the sequencing platform 145, and NGS or targeted NGS may be performed on a sample based on the request using the NGS unit 160. After the sequencing, the NGS reads or NGS data may be automatically sent to the server 135 through the network 120 for further processing. For example, the NGS data may be sent to the clinical genetic screening platform 115 to extract coverage information using tools 140 (e.g., the preprocessing unit 150). Variant data may be extracted or retrieved from the data repository 110 and sent to the clinical genetic screening platform 115 together with the NGS data. Additional information such as sensitivity profiles and population allele frequency information may also be extracted or retrieved from the data repository 110 using the tools 140. The extracted information together with the coverage information may be further processed using the rescue minimizer unit 155 to determine whether the sample needs to be rescued or if certain segments of the sample need to be rescued. The rescue information may be sent back to the sequencing platform 145 to perform confirmatory sequencing using the Sanger sequencing unit 165. The rescue information may also be communicated to the user of the client device 105 and the user may decide whether to perform the rescue. The Sanger sequencing data may be sent back to the server 135 or the clinical genetic screening platform 115 for subsequent analysis. For example, the NGS data and the Sanger sequencing data may be used together to determine if the sample comprises variants, or if a subject where the sample is obtained has developed a genetic condition (e.g., a disorder, a disease, or a cancer). The sample variant information or the disease diagnosis information may be transmitted to the client device 105 via the network 120. The data (e.g., the NGS data, the Sanger sequencing data, the variant data, sensitivity profiles, and/or population allele frequency information) may also be sent and stored in the data repository 110.

A data repository 110 is a data storage entity (or sometimes entities) into which data has been specifically partitioned for an analytical or reporting purpose. The data repository 110 may be used to store data and other information generated or used by the clinical genetic screening platform 115, the client device 105, and/or the sequencing platform 145. For example, one or more of the data repositories 110 may be used to store data and information to be used as input into the clinical genetic screening platform 115 for generating a final variant call report. In some instances, the data and information relate to genetic sequences (genomic, exomic, and/or targeted), high-confidence variants, information on variant type and clinical significance, population allele frequency, and other information used by the clinical genetic screening platform 115 when performing assay functions. The data repositories 110 may reside in a variety of locations including servers 135. For example, a data repository used by server 135 may be local to server 135 or may be remote from server 135 and in communication with server 135 via a network-based or dedicated connection of network 120. Data repositories 110 may be of different types or of the same type. In certain examples, a data repository 110 may be a database which is an organized collection of data stored and accessed electronically from one or more storage devices such as one or more servers 135. The one or more servers 135 may be configured to execute a database application that provides database services to other computer programs or to computing devices (e.g., client device 105 and clinical genetic screening platform 115) within the computing environment, as defined by a client-server model. One or more of these databases may be adapted to enable storage, update, and retrieval of data to and from the database in response to SQL-formatted commands or like programming language that is used to manage databases and perform various operations on the data within them.

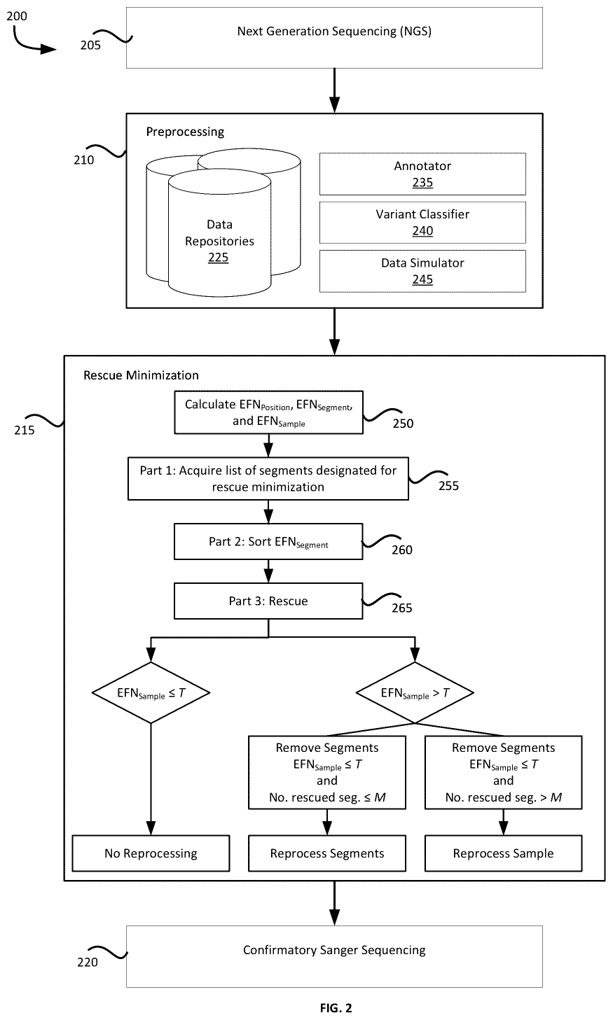

The clinical genetic screening platform 115 comprises a set of tools 140 for the purpose of analyzing and visualizing data (e.g., data stored in the data repository 110, data generated by the sequencing platform 145, or the data sent from the client device 105). The clinical genetic screening platform 115 is used to execute a process to designate high risk segments within a next generation sequencing sample for confirmatory testing of variant calls instead of all the segments in a sample to minimize the time and resources used in sample reprocessing. In the exemplary configuration depicted in FIG. 1, the set of tools 140 include two units: a preprocessing unit 150 and a rescue minimizer unit 155. The preprocessing unit 150 is capable of loading, processing, and saving data (e.g., accessed from the data repository 110) to be used by the preprocessing unit 150 itself and the rescue minimizer unit 155. The rescue minimizer unit 155 uses the processed data to identify a subset of high-risk segments in NGS read data for rescue and confirmatory sequencing by Sanger. In some instances, the clinical genetic screening platform 115 is used together with the sequencing platform 145 to: (i) generate NGS read data for a patient sample for regions of interest, (ii) obtain or identify variant information, population allele frequency information, and sensitivity profile using data obtained from the data repository 110, (iii) determine the expected number of false negative variant calls for the patient sample based on the NGS read data generated in (i) and the data obtained or identified in (ii), (iv) compare the expected false negative calls of the sample to a predetermined threshold, (v) designate a subset of segments for rescue, and (vi) perform confirmatory Sanger sequencing on the subset of segments designated for rescue. The NGS read data and the Sanger sequencing data are used to obtain an accurate variant call for the sample with improved accuracy and specificity, as described in detail with respect to FIG. 2. The clinical genetic screening platform 115 may reside in a variety of locations including servers 135. For example, a clinical genetic screening platform 115 used by server 135 may be local to server 135 or may be remote from server 135 and in communication with server 135 via a network-based or dedicated connection of network 120. The clinical genetic screening platform 115 may be of different configurations or of the same configuration. The one or more servers 135 may be configured to execute a discovery application that provides discovery services to other computer programs or to computing devices (e.g., client device 105) within the computing environment 100, as defined by a client-server model.

In various instances, server 135 may be adapted to run one or more services or software applications that enable one or more embodiments described in this disclosure. In certain instances, server 135 may also provide other services or software applications that may include non-virtual and virtual environments. In some examples, these services may be offered as web-based or cloud services, such as under a Software as a Service (SaaS) model to the users of client device 105. Users operating client device 105 may in turn utilize one or more client applications to interact with server 135 to utilize the services provided by these components (e.g., database and rescue applications). In the configuration depicted in FIG. 1, server 135 may include one or more components that implement the functions performed by server 135. These components may include software components that may be executed by one or more processors, hardware components, or combinations thereof. It should be appreciated that various different device configurations are possible, which may be different from computing environment 100. The example shown in FIG. 1 is thus one example of a computing environment (e.g., a distributed system for implementing an example computing system) and is not intended to be limiting.

Server 135 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 135 may include one or more virtual machines running virtual operating systems, or other computing architectures involving virtualization such as one or more flexible pools of logical storage devices that may be virtualized to maintain virtual storage devices for the server. In various instances, server 135 may be adapted to run one or more services or software applications that provide the functionality described in the foregoing disclosure.

The computing systems in server 135 may run one or more operating systems including any of those discussed above, as well as any commercially available server operating system. Server 135 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 135 may include one or more applications to analyze and consolidate data feeds and/or data updates received from users of client devices 105. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 135 may also include one or more applications to display the data feeds, data updates, and/or real-time events via one or more display devices of client devices 105.

The sequencing platform 145 is configured to perform sequencing tasks including NGS and Sanger sequencing. The sequencing platform 145 may be performed fully automatically with loaded samples, or performed semi-automatically with the help of a practitioner. As illustrated in FIG. 1, the sequencing platform 145 may include two units: an NGS unit 160 performing next-generation sequencing, and a Sanger sequencing unit 165. In some instance, the sequencing platform 145 may include additional units, such as a third-generation sequencing (TGS) unit (e.g., performing single molecule real-time (SMRT) sequencing and/or nanopore sequencing), a pyrosequencing unit, an Ion Torrent sequencing unit, and/or a sequencing by ligation (SOLiD) unit. In some instances, the NGS unit 160 is capable of performing the functions in the additional units.

NGS is a powerful technology that allows for the rapid sequencing of entire genomes or targeted regions of DNA or RNA. In some instances, the NGS unit 160 performs a nucleic acid extraction process to isolate high-quality DNA or RNA from a biological sample. This is followed by fragmentation, where the extracted nucleic acids are broken into smaller, more manageable pieces. This can be achieved through mechanical shearing, enzymatic digestion, or sonication. The fragmented DNA or RNA is then prepared for sequencing through the addition of sequencing adapters. These adapters are short, double-stranded DNA sequences that are ligated to the ends of the fragments, allowing them to bind to the sequencing flow cell and facilitate amplification. The NGS unit 160 may also perform library preparation, which involves further processing to ensure that the fragments are of the appropriate size and concentration for sequencing. This can include size selection, where fragments of a specific length are isolated using gel electrophoresis or magnetic beads. The prepared library is then quantified and quality-checked using techniques such as quantitative PCR (qPCR) or bioanalyzer assays to ensure that it meets the requirements for sequencing. In some instances, the wet-lab procedures are performed by a trained practitioner.

Once the library is ready, it can be loaded onto the sequencing platform 145 or the NGS unit 160. Different NGS platforms may have their own sequencing chemistries and technologies, generally involving the attachment of the library fragments to a solid surface, amplification to create clusters or colonies of identical sequences, and sequencing-by-synthesis or other methods to read the nucleotide sequence of each fragment. The sequencing process performed by the NGS unit 160 generates massive amounts of data (e.g., millions to billions of sequence reads), which can be then transferred to the server 135 or the clinical genetic screening platform 115 for analysis. In some instances, the NGS unit 160 or another component of the sequencing platform 145 may analyze, process, or manage the sequencing data. For example, bioinformatics tools and algorithms may be employed to process raw sequencing data, which includes base calling, quality control, read alignment, and variant calling. High-performance computing systems and cloud-based platforms are often used to handle the computationally intensive tasks of sequence alignment and data analysis. Additionally, specialized software pipelines are used to assemble the sequenced reads into complete genomes or to identify genetic variants. The integration of artificial intelligence and machine learning algorithms may be further adopted to enhance the accuracy and efficiency of data analysis, enabling the identification of novel genetic markers and potential therapeutic targets.

The Sanger sequencing unit 165 is configured to perform Sanger sequencing for determining the nucleotide sequence of DNA or RNA. The Sanger sequencing unit 165 is capable of synthesis of a complementary DNA strand using a single-stranded DNA template (or an RNA template), a DNA polymerase enzyme, and a mixture of normal deoxynucleotides (dNTPs) and chain-terminating dideoxynucleotides (ddNTPs). The ddNTPs are fluorescently or radioactively labeled and lack a 3′ hydroxyl group, which prevents further elongation of the DNA strand upon incorporation. By including a small proportion of ddNTPs in the reaction, a series of DNA fragments of varying lengths is generated, each terminating at a specific nucleotide. The resulting DNA fragments are then separated by size using capillary electrophoresis or polyacrylamide gel electrophoresis. In capillary electrophoresis, an electric field is applied to a capillary tube filled with a polymer matrix, which allows the fragments to migrate based on their size. Smaller fragments move faster through the capillary, while larger fragments move more slowly. As the fragments pass through a detector, the fluorescent or radioactive labels are detected, and the sequence of the DNA or RNA is determined by analyzing the order of the labeled fragments. The sequence data can also be sent to the server 135 or the clinical genetic screening platform 115 for analysis. In some instances, the Sanger sequencing data can be compiled and interpreted using the sequencing platform 145 to reconstruct the original DNA or RNA sequence, validate of sequences obtained from the NGS unit 160, or detect variants in the biological materials. Sanger sequencing remains a gold standard for its accuracy and reliability, particularly for smaller-scale sequencing tasks, diagnostic applications, and confirming genetic variations identified by other methods (e.g., the NGS method).

### III. Rescue Minimization Process

FIG. 2 shows a block diagram illustrating a clinical genetic screening assay 200 in accordance with various embodiments. As shown in FIG. 2, the clinical genetic screening assay 200 includes a Next Generation Sequencing (NGS) subsystem 205 (or NGS assay 205), a preprocessing subsystem 210, a rescue minimization subsystem 215, and a confirmatory Sanger sequencing subsystem 220 (or confirmatory Sanger sequencing assay 220). Each of the subsystems of the clinical genetic screening assay 200 may be interrelated and can be performed using one or more components or units of the computing environment 100 described with respect to FIG. 1. For example, the NGS assay 205 may be performed using the NGS unit 160 of the sequencing platform 145 of FIG. 1, the preprocessing subsystem 210 may be performed using the preprocessing unit 150 of the tools 140 of the clinical genetic screening platform 115 of FIG. 1, the rescue minimization subsystem 215 may be performed using the rescue minimizer unit 155 of the tools 140 of the clinical genetic screening platform 115 of FIG. 1, and the confirmatory Sanger sequencing assay 220 may be performed using the Sanger sequencing unit 165 of the sequencing platform 145 of FIG. 1. In some embodiments, one or more of the subsystems of the clinical genetic screening assay 200 or a portion thereof may be performed using a different environment or platform, or by a practitioner.

The goal of the clinical genetic screening assay 200 is to minimize the number of NGS samples and/or regions of interest (ROIs) of an NGS sample that need reprocessing by identifying smaller portions, or segments, of the samples that reside in the ROIs that are at a relatively higher risk (comparing to other portions or segments) of receiving false negative calls. In some embodiments, after performing NGS on a patient sample using the NGS assay 205, ROIs or genomic positions previously reported to have variants known to be highly associated with genetic conditions (e.g., pathogenic/likely pathogenic variants) or variants with high variant effect predictor (VEP) scores are evaluated using the rescue minimization subsystem 215 by determining the expected false negative (EFN) value, based on the read coverage and variant type at that particular location. The evaluation is further determined based on population allele frequency found in large biobanks (e.g., gnomAD) and a sensitivity profile, which are generated by the preprocessing subsystem 210. By taking the sum of all the putative variant positions per sample, the EFN value for the sample is determine and compared to a predetermine threshold of a tolerable false negative risk (e.g., “T” in FIG. 2) for that particular sample. If the EFN for the sample is greater than the threshold, segments with relatively higher false negative risk are removed so that the EFN of the sample is less than or equal to the threshold. The removed “high-risk” segments are then designated for confirmatory sequencing by Sanger so that a confident variant call is made. In the event that too many segments are removed, the whole sample may be designated for NGS resequencing or Sanger resequencing.

The NGS assay 205 allows for the rapid sequencing of entire genomes or targeted regions of DNA or RNA with high accuracy and throughput and is frequently used for routine diagnostics in genetic screening (e.g., newborn, carrier testing, prenatal diagnostic testing, predictive or predispositional genetic testing, and the like) for detecting variants strongly associated with genetic conditions. NGS encompasses a variety of high-throughput sequencing techniques, including sequencing by synthesis (e.g., Illumina Sequencing), semiconductor sequencing (e.g., Ion Torrent Sequencing), single-molecule real-time (SMRT) sequencing, nanopore sequencing, and ligation-based sequencing (e.g., SOLiD Sequencing). The NGS assay may be carried out by the sequencing platform 145 described with respect to FIG. 1. In some instances, the NGS assay 205 in FIG. 2 may use a process known as clonal amplification to amplify the DNA fragments of a patient sample and bind them to a flow cell. Then, a sequencing by synthesis method is used where fluorescently labeled nucleotides (A, C, G, T) compete for addition onto a growing chain based on the sequence of the template. A light source is used to excite the unique fluorescence signal associated with each nucleotide and the emission wavelength and fluorescence signal intensity determine the base call. Each lane in a flow cell can hold hundreds to millions of DNA templates, giving NGS its massively parallel sequencing capabilities. It should be understood that different NGS techniques may be used by the NGS assay 205 to provide sequencing data for subsequent processing and analysis.

NGS assay 205, can be applied in various sequencing techniques that include whole-genome sequencing (WGS), whole-exome sequencing (WES), targeted genome sequencing (TGS), and any other sequencing technique known to one skilled in the art. Typically for routine diagnostic genetic screenings, panels of genes or target ROIs known to have strong associations with the pathogenesis of disease and/or clinical relevance are used and processed by TGS. This method involves selecting a genetic panel that has probes complementary to the target regions of interest in the NGS sample. Additional approaches for TGS can also include hybridization capture or PCR amplification with target-specific probes followed by NGS, array-based hybridization using probes targeting exons, in-solution hybridization capture where biotinylated DNA or RNA molecules are used as bait to capture target regions, and the like.

ROIs can be predetermined based on their association with a specific disease or based on the clinical screening performed. For example, CFTR may be an ROI for cystic fibrosis, HBB for sickle cell anemia and beta-thalassemia, and FMR1 for Fragile X syndrome. In some embodiments, ROIs may be determined based on the coverage and the variants. For example, the clinical genetic screening assay 200 focuses on low coverage regions only in locations that contain a known pathogenic or likely pathogenic variant or where a variant could have a deleterious effect on a gene. In some instances, “low coverage regions” refer to the regions having an average sequencing depth of about or less than 40×, 30×, 20×, or 15×. In some instances, “low coverage regions” refer to the regions having a minimum (or maximum) sequencing depth of about or less than 40×, 30×, 20×, or 15×. In some instances, the standard of “low” depends on the specific type of variants to be detected. For example, in the scenario of detecting somatic mutations, a much higher coverage threshold (e.g., 250×) may be used.

The preprocessing subsystem 210 is responsible for performing the initial set of steps (e.g., loading data, preprocessing data, and saving data into data structures) to prepare the data (e.g., NGS read data, variant call datasets, population allele frequency data, and the like) for use in the rescue minimization subsystem 215. Both the preprocessing subsystem 210 and the rescue minimization subsystem 215 are part of the clinical genetic screening assay 200 framework comprising hardware such as one or more processors (e.g., a CPU, GPU, TPU, FPGA, the like, or any combination thereof), memory, and storage that operates software or computer program instructions (e.g., Application Programming Interfaces (APIs), Cloud Infrastructure, Kubernetes, Docker, TensorFlow, Kuberflow, Torchserve, and the like) to execute arithmetic, logic, input and output commands in order to control the storage, organization, and retrieval of data. In some instances, the preprocessing subsystem 210, rescue minimization subsystem 215, or both implement deployment of the data using a cloud platform such as Amazon Web Services (AWS), Google Cloud Platform (GCP), and Microsoft Azure. The preprocessing subsystem 210 can be performed using the server 135 or the tools 140 (e.g., the preprocessing unit 150) described with respect to FIG. 1. As shown in FIG. 2, the preprocessing subsystem 210 may include data repositories 225 and a set of sub-processors including an annotator 235, a variant classifier 240, and a data simulator 245.

The data repositories 225 (e.g., data repository 110 as described with respect to FIG. 1) are configured as databases that store sets of data that can be obtained from publicly available data sources (e.g., NCBI, ClinVar, gnomAD, Genome in a Bottle (GIAB), and the like), commercial sources (e.g., UK Biobank), and privately (e.g., in-house) processed data. Further, the data stored in the data repositories 225 may be accessed by any of the sub-processors within the preprocessing subsystem 210 for data annotation, classification, simulation, and the like. The types of data stored in data repositories 225 may include sequencing data, datasets with information on known variants such as their location, type, clinical relevance, and the like, high confidence benchmark variant datasets, population allele frequencies, and any other information that may be necessary.

Sequencing data may include sequencing information related to the whole genome, exomes, or specific regions of interest (e.g., regions selected to appear in gene panels). More specifically, sequencing of samples produce a large number of sequence reads (e.g., long reads (e.g., reads that are longer than 1,000 base pairs (bp)), and/or short reads (e.g., reads that are about 30 bp-300 bp)) deposited in a file with associated quality scores (e.g., FASTQ). These reads are typically aligned to a reference sequence, such as a reference genome, and the results are deposited in an alignment file (e.g., BAM). Variants are called based on the alignment and their properties relevant to the sequence (e.g., type of variant) are annotated and deposited in a variant file (e.g., variant call format (VCF)). In some instances, variant files are obtained from a database, and the data in the variant file can be further analyzed to determine what findings are clinically relevant and reportable to a health care provider to inform medical decision making.

In some instances, the sequencing data is for an exome sequencing project, where the sequencing data of target regions is extracted and the target regions may contain variants. The sequencing data can provide information such as read coverage, which describes the average number of reads that align to or “cover” known reference bases. Further, the read coverage can determine the degree of confidence in which a variant call can be made. For example, the higher the read coverage the more confident and accurate the variant call is. When the minimum read coverage is less than a predetermined threshold, an alternative sequencing method may be required to reprocess the segments to make sure that clinically relevant variants can be detected with high confidence in these segments.

As described herein, variants comprise naturally occurring alterations to the DNA sequence not found in the reference sequence. Information on variants can be accessed through public-private-academic archives that collect information pertaining to the relationships between human variations and phenotype (e.g., an observable characteristic such as an observable health status). Information on variants can also be accessed through commercial sources. The relationship may describe the clinical significance of variants, for example if a variant is benign, likely benign, variant of unknown significant, likely pathogenic, or pathogenic. Benign and likely benign variants refer to variants that either are not or are probably not responsible for causing disease where the degree of research varies from ample support to not enough support to be certain. Variants of unknown significance occur when it is unclear whether that variant is harmless or actually connected to a health condition. In other words, the variant lacks sufficient evidence to meet the criteria for likely pathogenic or likely benign. Similar to benign and likely benign variants, pathogenic and likely pathogenic variants are variants with either ample support for causing a disease, or probably cause a disease, but there is not sufficient evidence, respectively. In this context, “likely” refers to a variant as greater than or equal to 95-98% probability of being disease causing (with respect to likely pathogenic) or harmless (with respect to likely benign). As described herein, greater than or equal to 95-98% probability includes values 95, 96, 97, and 98. With regards to genomic location, variants reported in archives should be mapped to a reference genome so that their chromosomal location is documented.

In addition to clinical relevance, information on variant type can also be included in these archives or sources. Examples of variants include small variants (e.g., less than 50 base pairs) such as single nucleotide variants (SNVs), single nucleotide polymorphisms (SNPs), insertions and deletions (sometimes referred to as indels), structural variants (e.g., greater than 50 base pairs) such as insertions, deletions, chromosomal rearrangements (e.g., translocations and inversions), and copy number variations (CNVs). SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Further, variants can occur in both coding and non-coding regions of the genome and detected by WES, TGS, or WGS respectively.

In some instances, an application programming interface (API) is used to predict the effect of a variant on genes, transcripts, protein sequence, and regulatory regions and store this information in a dataset. An API connects computers or pieces of software to each other. In other words, it is a type of software interface that offers a service to other pieces of software. An API is often made up of different parts which act as tools or services that are available to the programmer to use and call that portion of the API. Typically, APIs are used to hide the internal details of how a system works and only shows the programmer the components they would find useful. For example, a programmer can input the coordinates and nucleotide changes of a specific variant and receive output describing the genes/transcripts affected, consequences of variant on protein sequence (e.g., gain/loss of stop codon, missense, frameshift, etc.), if there are known variants that match the input variant, and the like, but not be exposed to all the different sources the software used to obtain the output information.

In some cases, high-confidence variant call datasets (e.g., benchmark variant datasets) can be used for accurately classifying variants into groups. These datasets are specifically validated by various sequencing and variant calling technologies and/or obtained from a reliable source known for distributing or providing confirmed or benchmarked variant call data.

Datasets related to variant frequency within a population, otherwise known as population allele frequency, can be obtained from reliable sources that have aggregated large collections of human sequencing data (e.g., gnomAD). Population allele frequency refers to how common an allele is in a population and can be determined by taking the ratio of how many times a particular allele appears in the population to the total number of copies of the gene in a population. Often, population allele frequency data is used to discern rare variants that are more likely to be the cause of genetic disorders to be distinguished from more common and benign variants present across the human genome.

The annotator 235 loads and filters data obtained by the NGS assay 205 (e.g., the targeted NGS read data), datasets obtained from the data repositories 225 (e.g., variant datasets such as ClinVar datasets), and/or API generated variant predictor datasets (e.g., Ensemble Variant Effect Predictor (VEP) datasets). In some instances, sequencing or variant data obtained from the data repositories 225 is input into the annotator 235 to extract pathogenic and likely pathogenic variants. In some instances, the annotator 235 filters the input data or the extracted data to include variants in the target regions of interest. In some instances, variant data files are obtained from a database and run using a coded computer program. The coded computer program may be configured to extract pathogenic and likely pathogenic variants from input files. The coded computer program may be further configured to filter variants that are from targeted regions of interest. For example, the output data file provided by the coded computer program may be in a specific format (e.g., VCF format) including only those variants categorized as pathogenic or likely pathogenic and located on the target regions of interest. In some instances, the output data or data file include variant locations associated with the variants (e.g., hg19 coordinates).

In some instances, the annotator 235 also generates or predicts high impact variants. The term “high impact variants” refer to variants predicted to have a disruptive impact in the gene expression and/or associated protein, causing protein truncation, loss of function, or triggering nonsense mediated decay, or variants predicted to affect a sequence of its gene, transcript, protein, or any combination thereof. In some instances, the annotator 235 generates computationally predicted high impact variants using a coded computer program and/or through API. As for the API generated variant predictor dataset, API interfaces, such as VEP, make predictions on how likely a given variant is to have a disruptive impact in the gene expression or an effect on its corresponding gene, transcripts, protein sequence, and regulatory regions. In some instances, all SNVs for the target regions of interest are generated (e.g., using a custom-developed Python program) for prediction of how each potential variation might affect the gene or protein expression.

The annotator 235 may include a predictor model that predicts biological effects of variants (including functional consequences, allele frequencies, and/or related phenotypes) based on information obtained from the data repositories 225. In some instances, the annotator 235 ensembles a set of predictor models. Variants can be accessed by the VEP (programmed or input by a user) and VEP will output scores for each variant or determine if the variant satisfies a predetermined criterion of being high impact. In some instances, the annotator 235 keeps those variants that receive a high VEP score indicating they are more likely to have a clinical effect and filtering the variants that are not high impact variants. In some instances, the annotator 235 generates the computationally predicted high impact variants based on variants on the target regions of interest obtained from the data repositories 225. The variants may comprise different types of variants, or a single type of variants (e.g., SNVs).

Once all datasets have been loaded and filtered, annotator 235 can union or combine the segments with both the pathogenic/likely pathogenic variant dataset and the high impact variant dataset to generate a list of target ROI segments that contains either a pathogenic or likely pathogenic variant(s), a high impact variant(s), or both. In other words, the list of target ROI segments affirms that at a particular location in the ROI segment, a clinically relevant variant has previously either been reported/predicted or not. Using the list of target ROI segments in the clinical genetic screening assay 200 help improve the efficiency and reduce the cost associated with the clinical genetic screening. For example, those ROI segments that have no pathogenic or likely pathogenic variants previously reported and not include high impact variants do not need to be designated for rescue for the rescue minimization subsystem 215 and/or reprocessed by the confirmatory Sanger sequencing assay 220. On the other hand, those high-risk ROI segments that do overlap with the location of previously reported pathogenic or likely pathogenic variants and/or high impact variants can be candidates for rescue and processed by the rescue minimization subsystem 215 and the resulting high-risk ROI segment(s) are reprocessed by the confirmatory Sanger sequencing assay 220.

In some instances, the annotator 235 also extract or generate population allele frequency information from the data repositories 225. For example, population allele frequency information can be extracted or derived from a database comprising genomic data from a population or diverse populations (e.g., gnomAD). In some instances, the population allege frequency information includes a population allele frequency for each variant in the target ROIs. In some instances, a default allele frequency can be determined based on the population allele frequencies extracted or derived from the genomic data or variant data. Inclusion of population allele frequency for a variant at a particular genomic position can provide additional information about the probability of encountering that variant in clinical specimens, which can improve the accuracy of the rescue minimization process. The list of target ROI segments and/or the population allele frequency information can be further processed by the variant classifier 240, the data simulator 245, or input to the rescue minimization subsystem 215.

The variant classifier 240 loads the variant data (e.g., benchmark variant calls and high-confidence regions from GIAB) generated using a standard sequencing data analysis pipeline, for example, an NGS WES pipeline. As described above, an exemplary data analysis workflow involves aligning the FASTQ read file to a reference genome followed by variant calling. The variant classifier 240 may classify the called variants in a two-step process. First, variants with certain mappability scores (e.g., below 0.5) are grouped into variants located in low mappability regions or a low-map group. Then, the remaining variants are classified based on their zygosity (e.g., homozygous or heterozygous), variant type (e.g., SNV or indel), and variant length (e.g., short variants having 1-5 affected base pairs or long variants having 6-45 affected base pairs) producing, e.g., a heterozygous SNV group, a short heterozygous indels group, and a long heterozygous indels group. Variant classifier 240 can output a file for each variant group that lists out the variants that were categorized into each group.

The data simulator 245 performs the process of generating decimated samples with different coverage for calculating the probability a variant is detected at a specific location given the read coverage. Data simulator 245 loads the source FASTQ read files and the corresponding high-confidence truth variant sets from a reliable source (e.g., GIAB). During simulation, the raw reads from the original sample FASTQ files are titrated to range from 1-90% of the original read count. For example, and without limitation, if the original sample has a total of 200,000,000 reads, data simulator 245 will titrate the reads to range from 2,000,000-180,000,000 reads, where the 1% titration has 2,000,000 reads, the 2% titration has 4,000,000 reads, and the 90% titration has 180,000,000 reads. As described herein, a range from 1-90% includes values 1, 2, 3, 4, 5, 6, 7, 8, 9, 10, 20, 30, 40, 50, 60, 70, 80, and 90%, plus other intermediate values in this range (as needed). Once the reads have been titrated, NGS analysis and variant calling for each titration is performed and the simulated variant call is reported (“0” for NO and “1” for YES). A simulated variant call of NO indicates that the associated read coverage at that particular variant position was insufficient to detect the variant. In some instances, the simulated variant call and the corresponding coverage information for each high-confidence truth variant are combined for all the decimated samples to generate a final variant detection table. The high-confidence truth variants can be put into six groups. First, all variants with mappability score below a predetermined score (e.g., about 0.4, 0.5, 0.6, 0.7, or 0.8) are put in the low-mappability group. Then the remaining variants are split into five groups based on the variant Zygosity, type and length (e.g., HOMO, HET-SNV, HET-INDEL:1-5, HET-INDEL:6-15, HET-INDEL:16-45). The data in each of these groups can be used to generate group-specific sensitivity profile, which represents the probability a particular variant type is detected given a specific read coverage.

Data simulator 245 can process all the variants in each variant group (e.g., generated by the variant classifier 240) to generate a final variant detection sensitivity profile for each variant type. The sensitivity profile represents the probability a particular variant type is detected given a specific read coverage. Further, the sensitivity profile reflects the ratio of the number of times a variant is detected over the total number of times a particular coverage is observed for the variant type being assessed. In other words, if there was a total of 100 heterozygous SNVs in the heterozygous SNV group and at 10× coverage, 2 variants were detected, then the sensitivity for heterozygous SNV detection is only 2% (i.e., 2/100), indicating that 2% of the time when there is 10× coverage, the heterozygous SNV will be detected. Further, at 20× coverage, if 85 variants were detected, the sensitivity increases to 85%, indicating that 85% of the time when there is 20× coverage, the heterozygous SNV will be detected.

In some instances, sensitivity profiles can be smoothed by interpolation, for example by fitting a logistic regression curve, or a more complicated functional form. In some instances, for example, when there is a rapid increase in the probability of detecting a variant (a “fast” regime, as coverage increases from very low to moderate/high levels) or when there is a gradual increase in the probability of detecting a variant after the “fast” regime (a “slow asymptotic” regime, as coverage continues to increase, eventually approaching an asymptote near 100%), a single logistic regression curve may not fit the variant data well. The lower fitness may be caused by increased signal-to-noise ratio due to the fast accumulation of reads over a relatively narrow range of coverage. In such regimes, a piecewise logistic model p(coverage) described below may be used to smooth the variant data:

$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$

$$wherein :$$

$$\frac{{e}^{a + b \times T}}{1 + {e}^{a + b \times T}} = \frac{{e}^{c + d \times T}}{1 + {e}^{c + d \times T}}$$

The parameters a, b, c, d, and T can be determined by a maximum likelihood approach in which the logistic equation is imposed via a penalty.

During variant calling for the decimated samples, variants are identified by aligning the sample sequencing read file to a reference sequence and reporting out discrepancies where the sample sequencing file contains DNA alterations not found in the reference sequence. The initial variant calls are filtered to remove artifacts associated with sequencing and/or alignment (e.g., short-read alignment methods). Filtering can comprise any combination of different methods such as using benchmark datasets, population allele frequency datasets, manual review using data visualizing tools, setting quality criteria, and any other method known to one skilled in the art. Benchmark datasets (e.g., GIAB and Platinum Genome for human samples) are used to evaluate the accuracy of variant calls by comparing the variant in question to a set of benchmark ground truth variants. These variant call datasets have been validated using several different sequencing tools and variant calling tools, establishing their high confidence status. The high-confidence truth variant sets are used to construct the sensitivity profiles for each of the variant types based on variant calls from simulated samples described above.

Population allele frequency datasets, such as gnomAD, comprise large-scale sequencing projects of whole genome and exome profiles from around the world, and provide a reference set of allele frequencies for many different variants in the general population. This can be used as the probability that a particular variant is present in a sample and used in the calculation of EFN below (e.g., Equation (3)). Using population allele frequency can increase the overall accuracy and efficiency of the rescue minimization pipeline, as the risk of a false negative is lower for a low-coverage variant position with a low population allele frequency compared to a variant with the same coverage but a higher population allele frequency.

After all the data files have been processed by the preprocessing subsystem 210, they are used as input data files for the rescue minimization subsystem 215 to access. The purpose of the rescue minimization subsystem 215 is to further assess a subset of high-risk segments designated for rescue minimization and determine which of the high-risk segments need to be reprocessed by Sanger. In order to accomplish this, the rescue minimization subsystem 215 executes a series of pre-calculations at box 250 including calculating the EFN values for putative variant positions (EFN_{Position}), the EFN value for each of the ROI segment (EFN_{Segment}), and the EFN value of the sample (EFNₛₐₘₚₗₑ). The EFN value of the sample (EFNₛₐₘₚₗₑ) represents an aggregated risk of false negatives across all variant locations in a sample and can be used to determine if Sanger resequencing is needed. After the pre-calculations are completed, the rescue minimization subsystem 215 is divided into three parts: Part 1 (at box 255) acquires a list of segments designated for rescue minimization. Part 2 (at box 260) sorts the segments in descending order by their EFN_{segment} values and Part 3 (at box 265) performs segment rescue if necessary, based on a series of decisions. In some embodiments, Part 2 can be performed after Part 3 when it is determined that segments are to be removed from the sample.

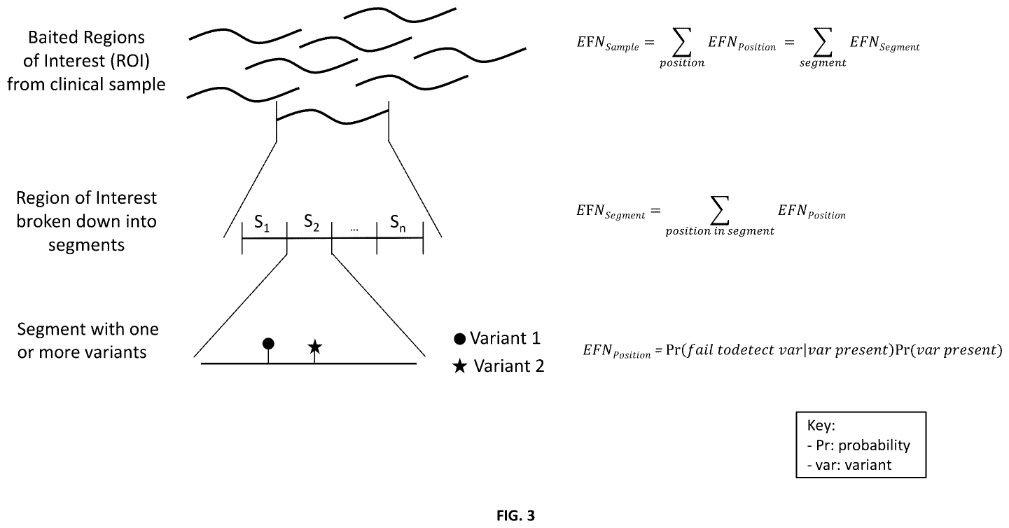

Before rescue minimization processing can begin, the EFN values for all putative variant positions, ROI segments, and samples are calculated as shown in box 250 and FIG. 3. FIG. 3 is an illustration of targeted NGS read data for ROIs that are screened for genetic testing in accordance with various embodiments. From top to bottom, a patient sample is prepared for targeted NGS of ROIs. The ROIs can be partitioned into smaller segments and each segment may contain one or more variants. The equations for calculating EFN_{Position}, EFN_{Segment}, and EFN_{Sample} are also provided for reference.

Initially, the rescue minimization subsystem 215 accesses the overlapped file generated from annotator 235 that contains the list of target ROI segments that may contain either a ClinVar pathogenic or likely pathogenic variant, or a high scoring VEP variant, or both. At each position where a variant has previously been reported, the expectation that a false negative variant call occurs, condition on the NGS read coverage at each location and probability that variant is present (based on the population frequency for that variant), is determined and referred to as the EFN_{Position} value. Taking the sum over all the putative variation locations yields the EFN_{Segment}, and the sum of all EFN_{Segment} yields the EFN_{Sample}. In more detail, the EFN_{Sample} can be described as follows: let xᵢ be a random variable where if xᵢ=1, then there is a false negative variant assessment in a sample at position i, otherwise xᵢ=0. The total number of false negative variant assessments in the sample, X, is X=x₁+, . . . , +x_{N}, where N is the number of pathogenic, likely pathogenic, or VEP=high positions in the ROI. With the notation Cᵢ=coverage at location i, Dᵢ=did detect a variant at position i, Fᵢ=did not detect a variant at position i, and Pᵢ=variant at position i is present, EFN_{Sample} can be described by:

$$E ( X \vert {C}_{1} = {c}_{1} , {C}_{2} = {c}_{2} , ... , {C}_{N} = {c}_{N} ) = E ( {x}_{1} \vert {C}_{1} = {c}_{1} ) + , ... , + E ( {x}_{N} \vert {C}_{N} = {c}_{N} ) \qquad Equation ( 1 )$$

$$E ( {x}_{i} \vert {C}_{i} = {c}_{i} ) = Pr ( {P}_{i} , {F}_{i} \vert {C}_{i} = {c}_{i} ) = Pr ( {F}_{i} \vert {C}_{i} = {c}_{i} , {P}_{i} ) Pr ( {P}_{i} )$$

The term Pr(Fᵢ|Cᵢ=cᵢ,Pᵢ) represents the probability that a variant is not detected at a specific location given the read coverage at that position and the variant type. The probability a variant is detected at a specific location given the read coverage and variant type at that position (i.e., Pr(Dᵢ|Cᵢ=cᵢ,Pᵢ)) was previously determined by data simulator 245. The rescue minimization subsystem 215 accesses the simulated data for each variant group and takes 1−Pr(Dᵢ|Cᵢ=cᵢ,Pᵢ) to find the Pr(Fᵢ|Cᵢ=cᵢ,Pᵢ) value for each position where a variant has previously been reported. In other words, the EFN_{Sample} can also be described as:

$${EFN}_{sample} = {\sum }_{position} {EFN}_{position} \qquad Equation ( 2 )$$

$$wherein ,$$

$${EFN}_{position} = Pr ( failure to detect variant \vert variant is present ) Pr ( variant is present ) = Pr ( {F}_{i} \vert {C}_{i} = {c}_{i} , {P}_{i} ) Pr ( {P}_{i} ) \qquad Equation ( 3 )$$

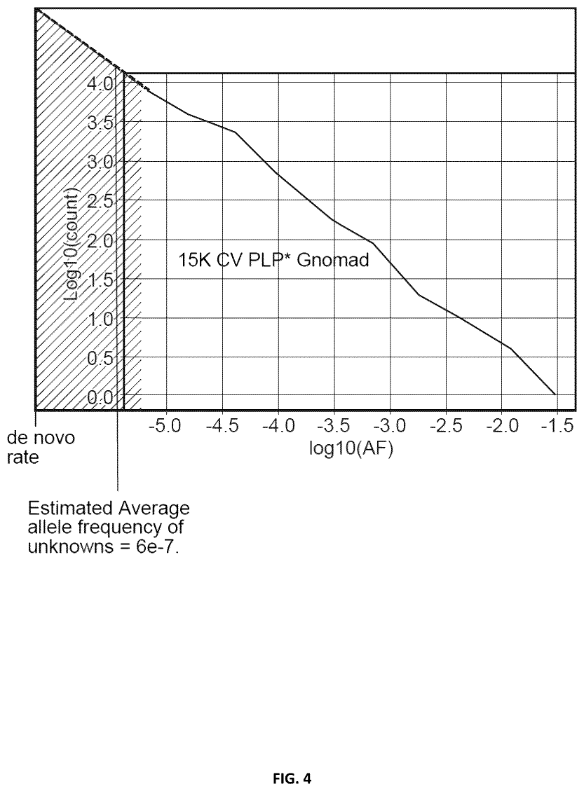

The term Pr(Pᵢ) or the probability a variant is present, is represented by the population frequency for the corresponding variant, provided that the variant is found in the reference database (e.g., gnomAD database). The reference database may also be used to determine allele frequency of the variants in the reference database and Pr(Pᵢ) denotes the corresponding population allele frequency (AF). For clinically relevant variants not found in the reference database, a statistical method (e.g., the power law) can be used to estimate the population AF. FIG. 4 shows a graph displaying the power law for AF counts in gnomAD in accordance with various embodiments. As shown in FIG. 4, the frequency of occurrence of an allele frequency in gnomAD follows a power law (solid line). An average allele frequency of a variant that has not been observed in gnomAD can be determined by extrapolating the power law beyond the smallest allele frequency in gnomAD (e.g., about 2E-6) until the spontaneous allele frequency of about 1.11E-8 (represented by the dashed line in FIG. 4). In some instances, normalizing the power law in the extrapolated range (e.g., [1.11E-8, 2E-6]) yields the AF density and the average AF can be determined based on the AF density. In some instances, the probability that a variant is presented is assessed for different variant classes (determined by the variant classifier 240), for example, the heterozygous (het) SNPs class, the het short indel class (e.g., length less than 5), the het long indel class (e.g., length greater than 5), and the class for variants in low mappability regions.

After all calculations for EFN_{Position}, EFN_{Segment}, and EFN_{Sample} are performed, the rescue minimization subsystem 215 can generate a table of all the segments that may contain one or more variants (e.g., either a ClinVar pathogenic or likely pathogenic variant, or a high scoring VEP variant, or both), the genomic locations of the putative variants, as well as the total EFN_{Segment} value and inputs into Part 1 at box 255 (see e.g., Table 2). At box 260, Part 2 sorts the segments in descending order, by their EFN_{Segment} value and Part 3 designates segments for reprocessing/rescue if necessary. The first step in the rescue is to determine if the sum over all EFN_{Segment} in a sample (EFN_{Sample}) is greater than a predetermined false negative threshold (T). In the event that EFN_{Sample}≤T, none of the segments in the sample are designated for reprocessing. Otherwise, if EFN_{Sample}>T, the rescue minimization subsystem 215 will remove the high-risk segments with the largest EFN_{Segment} value until EFN_{Sample}≤T. The removed, high-risk, segments are designated for rescue and reprocessed by another sequencing platform, such as the confirmatory Sanger sequencing assay 220. In some instances, when the number of segments needing rescued is larger than a maximum number of allowed rescues per sample (M), the entire sample will be designated for rescue/resequencing. The value of M can be determined by qualified medical personnel and may fluctuate based on the sample, clinical test code, or experience of the medical personnel. The rescue minimization approach effectively provides a guarantee on the maximum EFN of a sample to lower the cost associated with genetic screening, a guarantee that cannot be achieved using conventional approaches. The reduced number of low-coverage segment rescues also enables high throughput NGS analysis for genome- or exome-scaled clinical testing, which is also unavailable or not practical with the conventional coverage-based approaches.

The subset of high-risk ROI segments that are designated for rescue are sequenced by the confirmatory Sanger sequencing assay 220, which can be performed using the Sanger sequencing unit 165 described with respect to FIG. 1. Briefly, confirmatory Sanger sequencing assay 220 specifically utilizes chain-termination where specialized DNA bases (dideoxynucleotides or ddNTPs) are randomly incorporated into a growing DNA chain of nucleotides (A, C, G, T) generating different length DNA fragments. Capillary electrophoresis separates the fragments by size and a laser is used to excite the unique fluorescence signal associated with each ddNTP. The fluorescence signal captured shows which base is present at a given location of the target region being sequenced. In some instances, other confirmatory assays (e.g., digital PCR, quantitative PCR, mass spectrometry-based sequencing, etc.) can be used for the same or similar purposes (validate or verify results obtained from the NGS assay 205).

Wet-lab materials such as DNA polymerase (an enzyme that synthesizes the new DNA strand), a mixture of deoxynucleotides (dNTPs) and dideoxynucleotides (ddNTPs), primers (which provide a starting point for DNA synthesis), buffer solutions (which maintain optimal pH and ionic conditions necessary for the enzymatic reactions involved in the DNA synthesis process), and purification kits or reagents are essential for Sanger sequencing to ensure accurate and efficient DNA sequencing. By incorporating the rescue minimizing subsystem 215, the use of these wet-lab materials can be significantly optimized. This clinical genetic screening assay 200 reduces the need for extensive biological material rescue, meaning that less starting material is required to achieve reliable results. Consequently, this leads to a more efficient use of reagents and consumables, lowering overall costs and resource consumption. Additionally, the rescue minimizing subsystem enhances the overall screening process by reducing the time required for Sanger resequencing. This time efficiency not only accelerates the workflow but also allows for quicker turnaround times in obtaining sequencing results, which is crucial in clinical diagnostics and research settings. Thus, the integration of the rescue minimizing subsystem 215 offers substantial advantages in terms of cost savings, resource efficiency, and expedited sequencing processes.

The confirmatory Sanger sequencing assay 220 generates confirmatory read data so a confident variant call is made. Following reprocessing, the clinical genetic screening assay 200 generates a final report that uses a combination of the original NGS read data from the NGS assay 205 and the confirmatory read data from the confirmatory Sanger sequencing assay 220. The final report can provide information on the variants detected and their clinical significance, the type of variant and which base or bases are altered from the reference sequence, as well as which variants were reprocessed by the confirmatory Sanger sequencing assay 220.

### IV. Flowcharts

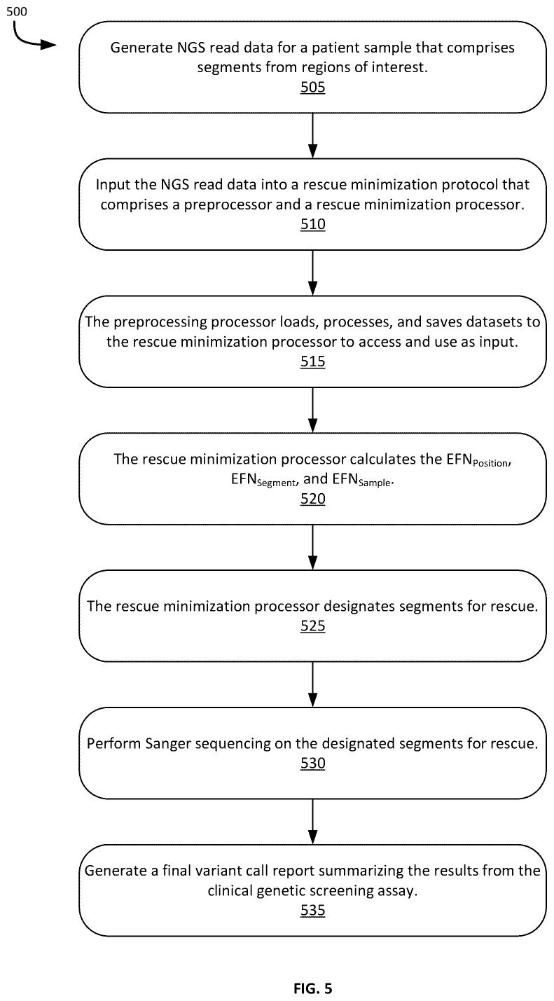

FIG. 5 is a flowchart illustrating process 500 for a clinical genetic screening assay to identify high-risk segments that need rescue and confirmatory Sanger sequencing for accurate variant detection in accordance with embodiments. The processing depicted in FIG. 5 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 5 and described below is intended to be illustrative and non-limiting. Although FIG. 5 depicts the various processing steps occurring in a particular sequence or order, this is not intended to be limiting. In certain alternative embodiments, the steps may be performed in some different orders, or some steps may also be performed in parallel.

At block 505, NGS read data for a patient sample is generated that comprises segments from regions of interest, wherein the segments contain one or more positions previously reported to have variants. The NGS read data may be generated through whole genome sequencing, whole exome sequencing, or targeted sequencing. For example, targeted NGS is performed using probes that are complimentary to probes on genetic panels that are configured to detect the alterations, or variants, in the DNA sequence in the one or more regions of interest. Importantly, the regions of interest are strongly associated with disease pathogenesis and/or have clinical relevance. In some instances, the NGS read data comprises segments with relatively low read coverage where an accurate variant call is not able to be made.

At block 510, results from the analysis of the NGS read data or targeted NGS read data is input into a rescue minimization protocol that comprises two processors: a preprocessing processor that generates input data based on the NGS read for the one or more regions of interest and a rescue minimization processor that identifies a subset of high-risk segments that need resequencing.

At block 515, the preprocessing processor loads various datasets, processes the datasets and saves them for use by the preprocessing processor itself and the rescue minimization processor. The types of datasets loaded can include a dataset of pathogenic and likely pathogenic variants from ClinVar, and/or a dataset of variants highly predicted to affect the sequence of its gene, transcript, protein, or any combination thereof generated by, e.g., the API Ensemble VEP, sequencing read data for a benchmark variant dataset, wherein the benchmark variant dataset comprising high confidence variants from, e.g., GIAB, and a population allele frequency dataset from, e.g., gnomAD.

Once the datasets are loaded, the preprocessing processor will begin processing the datasets into data structures that can be accessed and used as input files for the rescue minimization processor. For example, the preprocessing processor will overlap all the segments obtained from targeted NGS read data with the ClinVar dataset of pathogenic and likely pathogenic variants and the Ensemble VEP dataset of variants highly predicted to affect the sequence of its gene, transcript, protein, or any combination thereof to generate a final dataset that comprises a list of the segments that contain one or more positions previously reported to have either a pathogenic or likely pathogenic variant, a variants highly predicted to affect the sequence of its gene, transcript, protein, or any combination thereof, or both.

The preprocessing processor can also use the benchmark variant dataset comprising high confidence variants from GIAB to classify variants into groups. This can be accomplished in two steps where first, the high confidence variants are assessed based on their mappability score and, for example, when the mappability score is below 0.5, grouped those high confidence variants into a low mappability region variant group. Next, the remaining high confidence variants are classified based on zygosity (homozygous or heterozygous), variant type (SNV or indel), and variant length (short variants having 1-5 affected base pairs and long variants having 6-45 affected base pairs) to generate, for example, a heterozygous single nucleotide variants group, a short heterozygous indels group, and a long heterozygous indels group. The different variant groups can be used by the preprocessing processor to further generate variant sensitivity profiles for each group. The variant sensitivity profiles represent the probability of detecting a particular variant type (depending on the variant sensitivity profile used) given a specific read coverage at the variant's position and the type of variant present (e.g., Pr(Dᵢ|Cᵢ=cᵢ,Pᵢ) as described above). The profiles can be generated by titrating the read coverage of the high confidence variants, within the variant group being assessed, from 1-90% of their original coverage. Variant detection is simulated for the titrated reads by reanalyzing them with an NGS pipeline for variant calling. The output from this simulation process is a data file for each variant group that provides a list of all the variants within the corresponding variant group and whether the variants are detected at the specified read coverage. Simplistically, the data can be summarized as a ratio of the number of times a variant is detected over the total number of times a particular coverage is observed for the variant type being assessed. In other words, the ratio is the overall value of the variant type's sensitivity score. In practice, more sophisticated approaches are used in order to obtain smooth sensitivity profiles. Finally, because the probability of detecting a particular variant type given a specific read coverage at the variant's position and the type of variant present (e.g., Pr(Dᵢ|Cᵢ=cᵢ, Pᵢ) as described above) is known from the simulated data, it is then used to determine the probability that a variant is not detected given a specific read coverage at the variant's position and the type of variant (e.g., Pr(Fᵢ|Cᵢ=cᵢ, Pᵢ) as described in Equation 3) (e.g., by taking 1−Pr(Dᵢ|Cᵢ=cᵢ, Pᵢ) as described above). In some cases, the variant sensitivity profiles have already been generated, in which the preprocessing processor will load them.

At block 520, the rescue minimization processor access the final dataset (from block 515) that comprises the list of segments that contain one or more positions previously reported to have either a pathogenic or likely pathogenic variant, a variants highly predicted to affect the sequence of its gene, transcript, protein, or any combination thereof, or both, and performs a series of calculations to determine (i) the expected false negative (EFN) values for the one or more positions previously reported to have variants (e.g., EFN_{Position}=Pr(Fᵢ|Cᵢ=cᵢ, Pᵢ)Pr (Pᵢ) referenced with respect to Equation 3), (ii) the expected number of false negative (EFN) values for the segments (EFN_{Segment}), and (iii) the total EFN value of the sample (EFN_{Sample}) (e.g., Equation 2). To determine the EFN_{Position} values, the rescue minimization processor accesses the simulated data and, based on the coverage obtained from NGS and the population allele frequency associated with the variant in that position, generates EFN_{Position} values for all the positions in the final dataset (e.g., Equation 3). Accordingly, the sum over all EFN_{Position} values in a segment yields the EFN_{Segment} and the sum over all EFN_{Segment} values (or the sum overall EFN_{Position} values) yields the total EFN_{Sample} (e.g., Equation 1, Equation 2, and illustrated with respect to FIG. 3).

At block 525, the rescue minimization processor begins the process of designating a subset of high-risk segments for rescue, e.g., using a 3-part process. Part 1 receives a data table from block 520 that comprises all the segments that may contain one or more variants (e.g., either a ClinVar variant, a high scoring VEP variant, or both), the genomic locations of the putative variants, as well as the total EFN_{Segment} value. Part 2 sorts the segments in descending order, by their EFN_{Segment} value and Part 3 designates segments for rescue if necessary. Part 3 occurs as an iterative subprocess wherein each iteration comprises comparing the EFN_{Sample} value to a predetermined false negative threshold (T) and when the EFN_{Sample} value is greater than T, removing the high-risk segments with the largest EFN_{Segment} values until the EFN_{Sample} value is less than or equal to T. The removed high-risk segments are output and designated for Sanger sequencing. In some cases, the EFN_{Sample} value is already less than or equal to T and then none of the segments are designated for Sanger sequencing. Further, in the event that too many high-risk segments in a sample have to be designated for Sanger sequencing, which is based on a predetermined maximum number of allowed rescues per sample, the entire sample will be resequenced by NGS.

At block 530, Sanger sequencing is performed on the one or more high-risk segments designated for rescue, wherein the Sanger sequencing generates confirmatory read data and variant calls for the one or more regions of interest.

At block 535, a final variant call report is generated summarizing the results from the clinical genetic screening assay. The final report can contain information pertaining to the type of genetic screen received, the genomic location of the variant, variant type, nucleotide or chromosomal alteration detected, the clinical relevance of the variant (e.g., disease causing or benign), if Sanger sequencing was required, as well as any other information obtained from the clinical genetic screening assay.

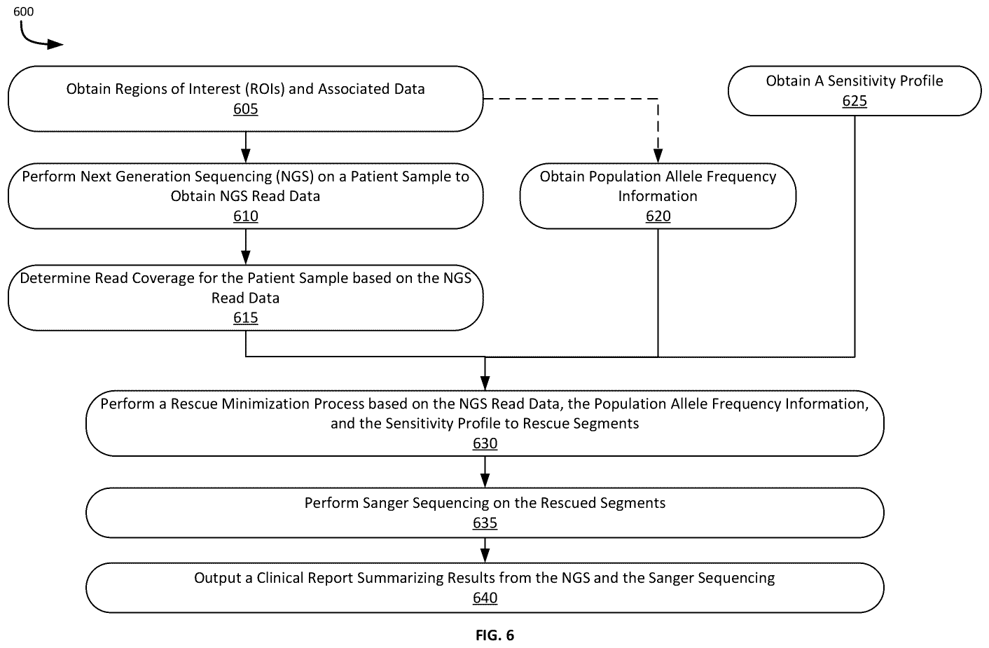

FIG. 6 shows a flowchart illustrating the rescue minimization pipeline 600 in accordance with various embodiments. The processing depicted in FIG. 6 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The rescue minimization pipeline 600 presented in FIG. 6 and described below is intended to be illustrative and non-limiting. Although FIG. 6 depicts the various processing steps occurring in a particular sequence or order, this is not intended to be limiting. In certain alternative embodiments, the steps may be performed in some different orders, or some steps may also be performed in parallel.

At block 605, the regions of interest (ROIs) and associated data are obtained. The ROI may be obtained by identifying specific regions or segments within a patient sample that are of particular relevance for diagnosis, research, or treatment of a disease (e.g., a genetic disease such as cystic fibrosis caused by CFTR gene mutations, or a genetic cancer such as breast cancer linked to BRCA1 and BRCA2 mutations). In some embodiments, the ROIs are determined based on clinical test panels. For example, the ROIs could be the specific genomic regions tested on genetic panels for disease-causing variants. As shown in FIG. 3, the ROIs can be partitioned into smaller segments where those segments may contain variants classified as pathogenic, likely pathogenic, or high impact (e.g., have a high VEP score). In some embodiments, each ROI corresponds to a segment Si shown in FIG. 3. The ROIs can be consecutive segments across a portion of a human genome, or nonconsecutive segments.

The “high impact variants” are variants known or predicted to have a disruptive impact in the protein, causing protein truncation, loss of function or triggering nonsense mediated decay.

In some embodiments, “high impact variants” are variants predicted to affect a sequence of its gene, transcript, protein, or any combination thereof. In some embodiments, the high impact variants are SNVs. For example, an analysis protocol may run through each base of a reference sequence for each ROI. Starting with the first base in the reference sequence, a computer script changes the reference base (e.g., “A”) to one of other three bases (“C,” “G,” and “T”) and analyzes the impact of each of these three changes. The process is iteratively repeated by moving on to the next base in the reference sequence and until all bases in the reference sequence have been analyzed and data for all “high impact variants” is stored. The associated data obtained at block 605 may include the segments/ROIs, genes associated with the segments and/or ROIs, genomic locations of ROIs and/or segments, and the like. In some embodiments, multiple ROIs and their associated data are obtained. In some embodiments, one single ROI and its associated data are obtained.

At block 610, next generation sequencing (NGS) is performed on a patient sample to obtain NGS read data. The patient sample may be obtained from a patient visiting a clinic or laboratories for determining if the patient has or is at risk for a specific disease. The NGS can be whole genome sequencing, whole exome sequencing, or targeted sequencing. In some embodiments, only NGS read data corresponding to the ROIs is obtained and further processed by the subsequent processes. In some embodiments, a targeted NGS is performed on the patient sample targeting the ROIs obtained at block 605. In some embodiments, the NGS data determines the ROIs.

At block 615, read coverage data is determined for the patient sample based on the NGS read data. In some embodiments, a read coverage is determined for each position of each ROI obtained at block 605 based on the NGS read data obtained at 610. Read coverage is a measure of the depth and can be used to evaluate the reliability of the NGS read data. Bioinformatics tools are used to process the NGS read data, determining the read coverage, and/or annotate sequence reads. For example, alignment tools such as BWA (Burrows-Wheeler Aligner) or Bowtie2 can map the NGS read data to a reference genome, and tools like BEDTools, SAMtools, GATK, MOSDEPTH, Pysam, Picard Tools, and the like can be used for filtering sequencing data, determining coverage data, and/or annotating or interpreting the sequencing or coverage data. Techniques disclosed herein can also be used to generate variant data.

In some embodiments, a subset of ROIs from the plurality of ROIs are excluded from the rescue minimization process at block 630. Each ROI of the subset of ROIs may have a read coverage below a predetermined minimum threshold, so that the Sanger sequencing is recommended to perform on the subset of ROIs. The predetermined minimum threshold can be 40×, 30×, 20×, 15×, 10×, or less than 10×. The predetermined minimum threshold may be determined based on the type of variant. In some embodiments, the predetermined minimum threshold is larger than 100× (e.g., 250×) when the variants are somatic variants.

At block 620, population allele frequency information is obtained. The population allele frequency information may include population allele frequency for the variants obtained at block 605 based on the ROIs. The population allele frequency information may comprise a population allele frequency for each variant that can be found in one of the ROIs obtained at block 605. In some embodiments, genomic data is obtained from large databases (e.g., gnomAD) and used as input to extract variant allege frequency in the ROIs. The population allele frequency information may also include genes, transcripts, or related information to the variants.

In the instances when the variant data obtained from the one or more databases (e.g., ClinVar or VEP) does not include a specific variant, a default allele frequency may be used as the population allele frequency for the specific variant. The default allele frequency may be determined based on a trend of variants in the ROIs based on the data in the one or more databases. For example, the default allele frequency may be generated by extrapolating a power law using the variant data obtained from the one or more databases. Details for determining population allele frequency can be found in the Examples section below.

At block 625, a sensitivity profile is obtained. The sensitivity profile is usually independent of the ROIs obtained at block 605, and also independent of the patient sample and its NGS read data obtained at block 610, so that it can be uniformly applied to different patients. The sensitivity profile includes information about a probability of each variant to be detected based on a read coverage and its variant type (e.g., SNVs, indels, or the like). The sensitivity profile may be obtained prior to or after the process performed at any of the blocks 605-620, or simultaneously with one or more of the processes. The sensitivity profile may be generated based on information obtained from human genomic research projects and databases. For example, Genome-in-a-Bottle (GIAB) HG001 and HG002 FASTQ read files are representative datasets of high-quality, well-characterized human genome sequences used as reference standards in genomic research. These files contain raw sequencing reads generated from NGS technologies, which capture the nucleotide sequences of DNA fragments from the HG001 and HG002 samples. HG001 (also known as NA12878) and HG002 are derived from individuals with known genetic backgrounds and have been extensively validated and annotated.

The HG001 and HG002 FASTQ read files can be used as the starting point to generate simulated samples with decimated coverage for calculating the probability a variant is detected at a specific location given the read coverage, in order to generate the sensitivity profile. Specifically, the raw reads from the HG001 and HG002 FASTQ read files can be titrated to generate decimated samples with 1-90% of the raw read count. Once the reads have been titrated, NGS analysis and variant calling for each decimated sample can be performed, and the GIAB truth variant detection status is reported (“0” for NO and “1” for YES). The GIAB truth variants are put into different groups. For example, all variants with mappability score below 0.50 are put in a low-mappability group, and remaining variants are split into five groups based on the GIAB truth variant Zygosity, type and length: HOMO, HET-SNV, HET-INDEL:1-5, HET-INDEL:6-15, HET-INDEL:16-45.

In some embodiments, the variant groups can be determined by a classification process. The classification process included steps of accessing sequencing read data for a benchmark variant dataset, wherein the benchmark variant dataset comprises the high confidence variants; determining a mappability score for each of the high confidence variants; grouping a first subset of the high confidence variants into a low mappability group, wherein each variant in the first subset has a mappability score of below 0.5; and grouping remaining high confidence variants into a heterozygous single nucleotide variants group, a short heterozygous indels group, or a long heterozygous indels group based on each variant's zygosity, variant type, and variant length. In some embodiments, the long heterozygous indels group includes mid long heterozygous indels each having 6-15 affected base pairs and long heterozygous indels each having 16-45 affected base pairs.

The binary data (e.g., the GIAB truth variant detection status) are used to generate the sensitivity profile. In some embodiments, the sensitivity profile is generated by interpolating the binary data using a logistic regression. In some embodiments, the sensitivity profile is generated by interpolating the binary data using a piecewise logistic regression model. In some embodiments, the piecewise logistic regressing model is

$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$

$$wherein :$$

$$\frac{{e}^{a + b \times T}}{1 + {e}^{a + b \times T}} = \frac{{e}^{c + d \times T}}{1 + {e}^{c + d \times T}}$$

wherein a, b, c, d, and T are predetermined parameters determined by a maximum likelihood approach in which the last equation is imposed via a penalty.

At block 630, a rescue minimization process is performed based on the read coverage data obtained at 615, the population allele frequency information obtained at 620, and the sensitivity profile obtained at 625. The rescue minimization process is to select segments (ROIs) for rescue based on expected false negatives (EFN) values for segments of the patient sample. The EFN value for each segment is determined based on positions and type of previously reported and clinically relevant variants (e.g., the pathogenic, likely pathogenic, or high impact variants obtained at block 605) in the segment, a population allele frequency of these variants, and the read coverage for these variant positions within the segment. A sample EFN value is determined based on EFN values of all segments of the sample. When the sample EFN value is greater than a predetermined threshold, a number of segments are rescued based on their contribution to it. A whole sample may be reprocessed if the rescue minimization process determines that too many rescues (e.g., more than 10 segments are rescued, or more than 20, 30, 40, 50, or 60 segments are rescued, or the number is over a threshold percentage, e.g., 10%, 20%, 30%, 40%, or 50%) are needed. The rescue minimization process can be performed using the rescue minimization subsystem 215 described with respect to FIG. 2.

In some embodiments, the rescue minimization process includes steps of determining an expected false negative (EFN) value for each segment of the plurality of segments based on the sensitivity profile and a population allele frequency of one or more variants in the segment, sorting the plurality of segments based on the EFN values, determining a sample EFN value for the patient sample based on the EFN values of the plurality of segments, determining if the sample EFN value is greater than a predetermined threshold, and when the sample EFN value is greater than the predetermined threshold, rescuing a number of segments from the plurality of segments based on the sorting, wherein a sum of the EFN values of remaining segments of the plurality of segments is less than or equal to the predetermined threshold. In some embodiments, the predetermined threshold is 1/5000, 1/10000, 1/15000, or 1/20000. The predetermined threshold may be determined based on a clinical or research need by a trained expert or practitioner.

At block 635, a Sanger sequencing (or the like) is performed on the rescued segments (ROIs) from the rescue minimization process. The Sanger sequencing generates confirmatory read data for the one or more rescued ROI for the patient sample.

At block 640, a clinical report is generated and output summarizing results from the NGS and the sanger sequencing. The clinical report can provide information regarding the variants detected and their clinical significance, the type of variants and which base or bases are altered from the reference sequence, as well as which variants were reprocessed by the Sanger sequencing. The clinical report can also include information pertaining to the type of genetic screening test received, the genomic location of the variant, the clinical relevance of the variant (e.g., disease causing or benign), as well as any other information obtained from the genetic screening test.

### V. Examples

The following examples are offered by way of illustration, and not by way of limitation.

High accuracy is critical for NGS-based clinical testing (e.g., genetic screening). In an NGS assay, the region of interest (ROI) is partitioned into segments. To ensure accurate variant calls, a segment whose minimum coverage is less than a pre-determined threshold, e.g., 20×, is “rescued” by an alternative method, such as Sanger sequencing. For large projects this can result in an un-manageable amount of effort. An alternative approach is disclosed herein that is both accurate and with minimized cost. As described above, an overall false negative (FN) risk for a sample is computed based on position and type of previously reported and clinically relevant variants in the ROIs, the population allele frequency of these variants, and/or the coverage for these variant positions within the ROIs. Samples whose FN risk is greater than a tolerance (e.g., a predetermined threshold) can have their highest risk segments reprocessed by another method (e.g., Sanger sequencing). The disclosed rescue minimization approach yields substantial savings compared to the conventional approaches based solely on coverage, while maintaining high accuracy for clinical testing.

The rescue minimization process achieves its goals by taking into account of a number of important factors. First, decimated data is generated from high confidence variant call benchmark datasets to create variant-type specific sensitivity profiles that reflect the probability a variant is detected conditioned on a specific read coverage and the variant type (e.g., heterozygous SNV, short heterozygous indels, long heterozygous indels, and low mappability region variants). Second, population allele frequency data is generated from larger databases to provide an accuracy variant frequency estimation. The rescue minimization process then combines the variant-type specific sensitivity profiles, the population frequency of the variant, and NGS read coverage data from a patient sample, to assess the risk of a false negative variant call being made in the segments of the patient sample. When the total FN risk of a sample is too high, the highest risk segments of the sample are designated for confirmatory sequencing by Sanger (i.e., are “rescued”). In addition, the disclosed rescue minimization approach ensures that the FN risk of every patient sample is bounded by a predetermined threshold.

When exome sequencing assays are used in clinical laboratories, frequently there are multiple ROIs that clinicians prioritize when determining if a patient has or is at risk for a specific disease. For example, the ROIs could be the specific genomic regions tested on genetic panels for disease-causing variants. Further, the ROIs can be partitioned into smaller segments where those segments may contain variants classified as pathogenic, likely pathogenic, or have a high VEP score. See FIG. 3 for an illustration of components comprising a targeted NGS sample. Accurate variant calling is dependent on sufficient read coverage during sequencing, where those segments with insufficient read coverage require reprocessing with Sanger sequencing. Current practice is to reprocess the entire set of low-coverage segments in the sample, despite only a small fraction of these segments with the possibility of having clinically significant variant(s). To minimize the number of segments in a sample being reprocessed, a novel process aimed at only reprocessing the high-risk segments that have a high probability of containing a false negative variant call was developed and validated.

### Data Acquisition and Preprocessing

Experimental data can be acquired by sequencing samples obtained from clinical testing (including genetic screening) using the NGS assay 205 described with respect to FIG. 2, and preprocessed by the preprocessing subsystem 210 to generate variant data files. Experimental data can also be acquired from publicly available databases, private databases, or commercial databases. For example, to validate the accuracy and efficiency of the disclosed rescue minimization pipeline (e.g., the rescue minimization subsystem 215), ClinVar data files were downloaded from NCBI. The variant data files were processed to extract pathogenic and likely pathogenic variants and convert to VCF format with hg19 coordinates. Variants in the target regions of interest were extracted with bedtools (Quinlan, A. R. and Hall, I. M. BEDTools: a flexible suite of utilities for comparing genomic features. Bioinformatics 2010; 26(6):841-842, incorporated by reference herein) and used as one of the inputs for the rescue minimization pipeline.

Additional variant data can be included as inputs for the rescue minimization pipeline. For example, SNVs obtained from the variant data files can be combined for targeted regions and used to generate variants that are predicted to have “high impact” to chromatin or gene expression. Features that are considered having “high impact” to the chromatin or gene expression may include (i) a feature ablation whereby the deleted region includes a transcript feature; (ii) a splice variant that changes the 2-base region at the 3′ end of an intron; (iii) a splice variant that changes the 2-base region at the 5′ end of an intron; (iv) a sequence variant whereby at least one base of a codon is changed, resulting in a premature stop codon, leading to a shortened transcript; (v) a sequence variant which causes a disruption of the translational reading frame, because the number of nucleotides inserted or deleted is not a multiple of three; (vi) a sequence variant where at least one base of the terminator codon (stop) is changed, resulting in an elongated transcript; (vii) a codon variant that changes at least one base of the canonical start codon; (viii) a feature amplification of a region containing a transcript; (ix) a sequence variant that causes the extension of a genomic feature, with regard to the reference sequence; and (x) a sequence variant that causes the reduction of a genomic feature, with regard to the reference sequence. In some embodiments, the first eight features are considered to have “high impact.” In some embodiments, any combination of the ten features and/or additional features may be considered. In some instances, the term “high impact variant” refers to a variant that is assumed to have disruptive impact in the protein, causing protein truncation, loss of function or triggering nonsense mediated decay. A “moderate impact variant” is a non-disruptive variant that might change protein effectiveness, and a “low impact variant” is a variant that is assumed to be mostly harmless or unlikely to change protein behavior. In some embodiments, a programmed computer code is run to generate the high impact variants.

### Population Allele Frequency Determination

Population allele frequencies may be used by the rescue minimization pipeline to determine false positive variants. Different techniques can be used to determine the population allele frequencies. For example, sequencing technologies such as whole genome sequencing (WGS) and targeted genotyping arrays are used to identify genetic variants across a population. Once variant data is obtained, bioinformatics tools (e.g., PLINK, VCFtools, and custom scripts in programming languages like Python or R) are employed to process and analyze the variant data. These tools can count the occurrences of each allele at specific loci across all individuals in the population. Statistical methods are then used to calculate the allele frequencies by dividing the count of each allele by the total number of alleles observed at each locus. Additionally or alternatively, databases like the 1000 Genomes Project and gnomAD can be used to provide population allele frequencies.

For example, exome and/or genome data can be obtained from gnomAD and bioinformatic tools such as Tabix can be used to extract allele frequency information from the obtained data. Other information such as gene symbol, transcript name, chromosome identifier, position of the variant on the chromosome, variant identifier, reference base and alternate base, and variant quality may also be extracted based on the obtained data. In some embodiments, only allele frequency information for the variants in the target regions is extracted. In some embodiments, the population allele frequency for each variant obtained from the exome dataset and genome dataset is combined to obtain the final population allele frequency.

As shown in FIG. 4, gnomAD includes allele frequencies for a substantial number of genetic variants. However, gnomAD does not encompass population allele frequencies for all possible variants, and might miss rare or novel variants not present in the aggregated datasets it uses to generate the population allele frequency. The present application discloses techniques for generating a default allele frequency for variants that are not presented in gnomAD (or other databases/sequencing techniques). The default allele frequency can be used as the population allele frequency for a variant whose population allele frequence information is missing from the sequencing data or the databases. The default allele frequency can be referred as an average allele frequency of variants that have never been seen. Details regarding generating the default allele frequency can be found in the rescue minimization process section above, e.g., by extrapolating a power law beyond the smallest allele frequency in gnomAD.

### Mappability Score Determination

Mappability scores can be used to group variants prior to the rescue minimization analysis. The mappability score for each segment can be calculated as the ratio of the coverage of uniquely mapped reads to the total coverage from all reads. In some embodiments, the mappability scores were calculated for all samples in a flowcell with multiple samples (e.g., about 190 samples), and the median mappability score for each segment can be used as the final mappability score for that segment in a combined mappability score table. The mappability score determination can be applied to high interest regions or challenging regions (e.g., clinically relevant regions) requiring special treatment, by including an additional “RescuePolicy” column to a segment to indicate if that particular segment should be excluded from the rescue minimization analysis, meaning that this segment should be rescued if the segment coverage is below a pre-determined coverage threshold.

Variant Detection with Decimated Coverage

Techniques can be implemented to generate simulated samples with decimated coverage (e.g., deliberate reduction in sequencing depth). The decimated coverage data can also be simulated for producing sensitivity profiles described below. The simulation can be performed using the data simulator 245 described with respect to FIG. 2. For example, after obtaining raw read files from a database (e.g., the GIAB HG001 and HG002 FASTQ read files), the raw reads were titrated to generate decimated samples, e.g., with 1-90% of the original read count. NGS analysis and variant calling were performed for each decimated sample and GIAB truth variant calls were reported (e.g., “0” for NO and “1” for YES). A GIAB truth variant call of NO indicates that the associated read coverage at that particular variant position was insufficient to detect the variant in a decimated sample. The variant detection status and the corresponding coverage info for each GIAB truth position are combined for all the decimated samples to generate a final variant detection table. The GIAB truth variants are then put into different groups (e.g., six groups). First, all variants with mappability score below 0.50 are put in the low-map group (see “Mappability Score Determination” described above). Then the remaining variants are split into five groups based on the zygosity, type and length of the GIAB variant: HOMO, HET-SNV, HET-INDEL:1-5, HET-INDEL:6-15, HET-INDEL:16-45. The data in each of these groups are then used to generate group-specific sensitivity profile, which represents the probability a particular variant type is detected given a specific read coverage.

### Sensitivity Profile Modelling

The sensitivity profile can be generated by the data simulator 245 described with respect to FIG. 2. The sensitivity profile can be generated by smoothing the variant data. The smoothing can be performed by interpolation using a logistic regression curve. In some instances, the smoothing can be performed by interpolation using a piecewise logistic model based on the coverage. For example, the piecewise logistic model p(coverage) described above can be used as the model for smoothing.

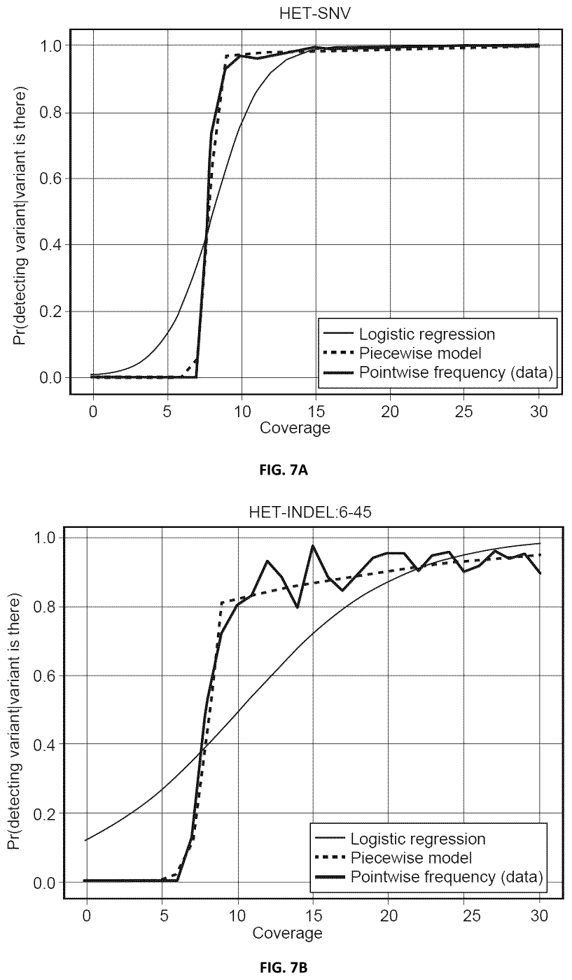

FIGS. 7A and 7B illustrate relationship between read coverage and probability of detecting the variant, and different smoothing models to simulate the relationship, in accordance with various embodiments. As shown in FIGS. 7A and 7B, the three curves in each for the HET-SNV and HET-INDEL:6-45 cases present the pointwise probability estimated from the data, calculated as the fraction of is for each coverage, the fit to the data of a logistic curve, and of the piecewise logistic model. FIGS. 7A and 7B illustrate that the piecewise logistic model aligns more closely with the pointwise probability than a single logistic regression. In some instances, due to the limited number of measurements for each coverage, the number of accurate decimal digits in the model can be limited and the probability for higher coverage regions were rounded up to 1.

### Tradeoffs

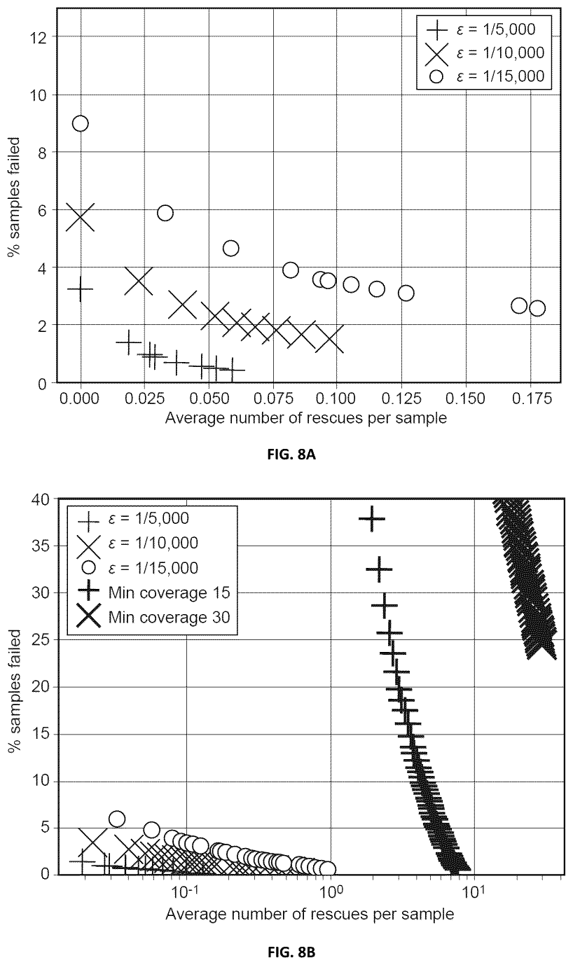

The rescue minimization pipeline was also tested on autosomes in 9 flowcells, encompassing a total of 1,645 samples, across different values of the false negative risk threshold (F). The amount of reprocessing required by the rescue minimization algorithm was analyzed and compared to the cost of the conventional coverage-based approach. As the allowed maximum number of rescues per sample (M) increases, the percentage of the samples that requires reprocessing decreases. Conversely, the average number of rescues per sample increases. This tradeoff is illustrated in FIG. 8A for P values of 1/5000, 1/10000, and 1/15000, with M ranging from 0 to 10. Table 1 provides the data used to generate the tradeoff curves. It is illustrated in FIG. 8A that as the threshold P becomes smaller the curves shift upwards and to right, indicating an overall increase in the number of rescues.

Similar tradeoff curves can be generated for comparing with the cost of the conventional coverage-based approach, which is based on rescuing every segment with minimum coverage less than a specified threshold. FIG. 8B shows the three tradeoff curves from FIG. 8A alongside the tradeoff curves for the conventional coverage-based approach using minimum coverage 15 and 30. It is evident that the disclosed rescue minimization approach is superior, requiring significantly less reprocessing.

TABLE 1

| | 1/5000 | | | | | |
|---|---|---|---|---|---|---|
| | | Average | 1/10000 | | 1/15000 | |
| Max | | rescue | | Average | | Average |
| rescue | % failed | per | % failed | rescue per | % failed | rescue per |
| per sample | samples | sample | samples | sample | samples | sample |
| 0 | 3.25 | 0 | 5.74 | 0 | 9 | 0 |
| 1 | 1.38 | 0.02 | 3.53 | 0.02 | 5.88 | 0.03 |
| 2 | 0.97 | 0.03 | 2.7 | 0.04 | 4.64 | 0.06 |
| 3 | 0.9 | 0.03 | 2.28 | 0.05 | 3.88 | 0.08 |
| 4 | 0.69 | 0.04 | 2.08 | 0.06 | 3.6 | 0.09 |
| 5 | 0.69 | 0.04 | 1.94 | 0.07 | 3.53 | 0.1 |
| 6 | 0.69 | 0.04 | 1.8 | 0.08 | 3.39 | 0.11 |
| 7 | 0.55 | 0.05 | 1.66 | 0.09 | 3.25 | 0.12 |
| 8 | 0.48 | 0.05 | 1.52 | 0.1 | 3.11 | 0.13 |
| 9 | 0.42 | 0.06 | 1.52 | 0.1 | 2.63 | 0.17 |
| 10 | 0.42 | 0.06 | 1.52 | 0.1 | 2.56 | 0.18 |

### Reliability Analysis

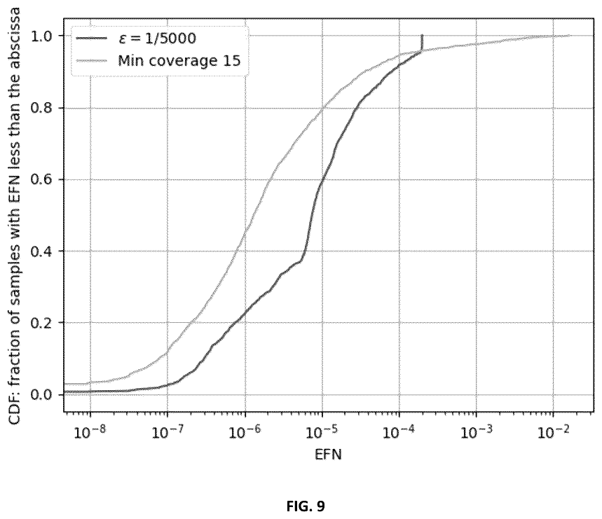

The disclosed rescue minimization approach also effectively controls the maximum expected false negative (EFN) values in sample segments, comparing to the conventional coverage-based approach. FIG. 9 illustrates the cumulative distribution (CDF) of the resulting EFN values for all the samples in the rescue minimization approach with F= 1/5000, and the coverage-based approach with minimum coverage of 15×. For the rescue minimization approach, all EFN values are less than or equal to 1/5000, with most samples having much lower EFN values. For instance, the 90th percentile EFN value is about 8.2E-5, which is less than 1/10000. On the right side of the curve, there is a noticeable vertical jump around 1/5000. This jump corresponds to the small fraction of samples that required rescue to ensure the sample EFN is always less than or equal to 1/5000.

For the conventional coverage-based approach with a minimum coverage of 15, the distribution of EFN values is shifted to the left. This is expected because the conventional strategy rescues many more segments, thereby reducing the total EFN of the sample. Only segments with minimum coverage greater or equal to 15 contribute to the EFN in this case. A significant drawback of this approach, beyond its high cost, is that it does not provide a guarantee on the maximum EFN across all samples. As shown in FIG. 9, 4.4% of the samples have an EFN value greater than 1/5000, with a maximum EFN of 1/62. This contrasts with the rescue minimization approach, which is designed to control the maximum EFN.

In some instances, a hybrid approach may be performed that combine the techniques in the disclosed rescue minimization approach and the coverage-based approach. For example, segments with coverage below the minimum threshold are selectively rescued based on segment specific criteria, and the remaining segments are then processed using the rescue minimization pipeline. This hybrid approach may be performed to focus on segments containing very high-risk variants. It also guarantees that the EFN for the entire sample remains within the desired threshold.

### Overview of Validation Process

The program that performs the rescue minimization was validated for correctness using standard approaches, such as unit testing. The code was partitioned into small units such that each unit of code was expected to perform a transformation of its input data into an output. The validation procedure involves first testing each unit of code separately using one or more of the following strategies:

Strategy 1: Compare the output data of each unit of code to output data that was determined by another method, often manually, and check that each unit of code operates correctly.

Strategy 2: Write another unit of code that tests if the output is consistent with the input. For example, the original unit of code will transform input data into output data, while the testing unit of code will retransform the output data back into the input data. Only when the original unit of code and the test unit of code are correct is the method validated. This approach is often used for units of code that load data into data-structures after performing a transformation.

Strategy 3: Incorporate additional statements into some units of code that tests if the expected assertions hold.

The rescue minimization process has two subprocesses (described in detail in FIG. 2). The first subprocess (e.g., the preprocessing subsystem 210 described with respect to FIG. 2) loads all the input data into internal data-structures. Examples of input data include the locations of variants, variant type, allele frequency, coverage at each location in the region of interest, variant sensitivity curves, etc. The second subprocess (e.g., the rescue minimization subsystem 215 described with respect to FIG. 2) performs rescue of segments for the set of segments that were designated for rescue minimization. For segments that were designated as rescue candidates, the second subprocess also determines which need to be rescued.

### Processor 1: Validation of Preamble Routines and Data Structure

The preamble routines of processor 1 are the initial steps taken before rescue minimization is assessed. These steps include loading input data (e.g., targeted NGS datasets, ClinVar variants, VEP datasets, GIAB dataset, genomAD datasets, variant sensitivity curves, and the like), preprocessing the input data, and saving the data into data structures for use in the main rescue minimization routine. Below is a list of computational functions used to validate the output of other units of code and the data structures they created. The validation process of processor 1 utilized strategy 2 described above.

The first validation function used was validate_load_roi. This function tests that (i) the positions of all segments were in the ROI, (ii) all segments were included in the rescue minimization program's data structure, (iiii) segments whose variant positions were ignored were not included in the ROI, and (iv) the intersection between the segments set and the coverage segments set was empty, wherein the coverage segment set comprises segments that had previously been designated for rescue by former methods based on minimum read coverage.

The validate_loc2minimize_segments function was used to (i) verify that every variant location in every segment maps back to its corresponding segment in the loc2minimize_segment data structure, and (ii) verify that each variant location in the ROI is found in the loc2minimize_segment data structure and that the segments the variants map back to are also contained in the loc2minimize_segment data structure.

The purpose of the validate_group_cov2prob function was to confirm that a table comprising the sensitivity curves of the variant groups: ‘LOW-MAP:score-0.50’, ‘HET-INDEL:1-5 bp’, ‘HET-INDEL:6-45 bp’, and ‘HET-SNV’ was loaded properly.

The validate_load_variants function was used to (i) verify that ‘LOW-MAP:score-0.50’, ‘HET-INDEL:1-5 bp’, ‘HET-INDEL:6-45 bp’, and ‘HET-SNV’ were the only variant types included in the variant table, and (ii) confirm that all the variants in the ROIs identified in the loc2minimize_segment data structure and the minimize_segs data structure associated with the correct segments.

To confirm that all data information from the genomAD database were loaded correctly, the Test_load_gnomad function was used. This function would print out the variant ID and allele frequency values, where the variant ID had to be displayed as ID=chrom:location:ref:alt and the allele frequency was reported as floating point numbers.

The validate_add_af function verified that the allele frequencies for those variants found in gnomAD were consistent with the gnomAD database, while the variants not found in gnomAD reported the default allele frequency.

The validate_location2variants function was used to verify that the information pertaining to all the pathogenic/likely pathogenic and VEP=high variants was present in the location2variants data structure.

Finally, the last validation function was validate_load_sample, which (i) verified that a value for read coverage at each position in the ROI was provided, and (ii) verified that a value for read coverage across all segments is defined as ‘Coverage’.

### Processor 2: Validation of Main Rescue Minimization Routine

The rescue minimization routine is partitioned into three parts: Part1 evaluates the data structure segment2loc_total which provides for each segment the list of locations that have one or more variants, and the total value of EFN at each such location. Part2 sorts the segments by the sum of EFN from all variant locations in each segment. Part3 performs rescue if necessary. Below is a list of computational functions used to validate the rescue minimization routine.

The validate_process_part1 function utilized strategy 1 described above, where a small amount of the output from the expected_segment2loc_total function was verified by hand. As shown in Table 2, the expected results matched the computational results. Further, the provided segments (HBB, HEXA, and NCOLN1) are examples of segments that contain 2 or less variants.

TABLE 2
Validation of Part 1 of the Rescue Minimization Routine.

| Segment | Code EFN | Manual EFN |
|---|---|---|
| HBB\|NM_000518\|IGR\|CCDS7753.1\|HBB_190_L\|GENESEQ | 5.999999999972694e−12 | 5.999999999972694e−12 |
| HEXA\|NM_000520\|I01\|CCDS10243.1\|HEXA_82_L\|GENESEQ | 5.999999999972694e−12 | 5.999999999972694e−12 |
| MCOLN1\|NM_020533\|IGR\|CCDS12180.1\|MCOLN1_13_L\|GENESEQ | 1.018824e−06 | 1.018824e−06 |

The next computational function used was validate_process_part2, which verified that the segments were sorted, in descending order, by the sum of all EFN_{Position} values (or EFN_{Segment}) from all their variants. This validation was accomplished using strategy 2 described above.

Finally, the validate_rescue function uses strategy 2, described above, to confirm that the input of the test unit of code is the list of segments that were selected for rescue. This process was achieved by (i) verifying that the EFN value of each segment that was designated for rescue was concordant with the EFN calculated by the Rescue Minimization Routine, (ii) verifying that the segments with the highest EFN among all segments were rescued, (iii) verifying that after rescue, the EFN of the sample was less than or equal to the threshold, and (iv) verifying that the number of segments rescued is the smallest possible number.

Three (3) samples are shown in Tables 3-5 as examples of the validation process described for the rescue minimization routine. In all 3 examples, the threshold (T) is set to 1/5,000. Each table shows the top segments in the list of segments ordered in decreasing order of EFN and the before and after rescue EFN value.

TABLE 3
Sample 1 Validation of Rescue Minimization Routine
Sample: 2228799080350_481893
Sample EFN before rescue: 3.4868e−04
Sample EFN after rescue: 1.8737e−04

| Segment | Rank | EFN | Rescued |
|---|---|---|---|
| IDUA\|NM_000203\|X09\|CCDS3343.1\|NA\|GENESEQ | 1 | 1.06E−04 | YES |
| NAGLU\|NM_000263\|X01\|CCDS11427.1\|NA\|GENESEQ | 2 | 5.51E−05 | YES |
| TSEN54\|NM_207346\|X01\|CCDS11724.1\|NA\|GENESEQ | 3 | 4.18E−05 | NO |

TABLE 4
Sample 2 Validation of Rescue Minimization Routine
Sample: 2229399077280_481893
Sample EFN before rescue: 2.5836e−04
Sample EFN after rescue: 1.5308e−04

| Segment | Rank | EFN | Rescued |
|---|---|---|---|
| NAGLU\|NM_000263\|X01\|CCDS11427.1\|NA\|GENESEQ | 1 | 5.65E−05 | YES |
| SGCB\|NM_000232\|X01\|CCDS3488.1\|NA\|GENESEQ | 2 | 4.88E−05 | YES |
| TSEN54\|NM_207346\|X01\|CCDS11724.1\|NA\|GENESEQ | 3 | 4.27E−05 | NO |

TABLE 5
Sample 3 Validation Routine Minimization Routine
Sample: 2229399077640_481893
Sample EFN before rescue: 2.7226e−04
Sample EFN after rescue: 1.6943e−04

| Segment | Rank | EFN | Rescued |
|---|---|---|---|
| GJB2\|NM_004004\|1\|CCDS9290.1\|GJB2_1\|GENESEQ | 1 | 5.90E−05 | YES |
| TSEN54\|NM_207346\|X01\|CCDS11724.1\|NA\|GENESEQ | 2 | 4.38E−05 | YES |
| SGCB\|NM_000232\|X01\|CCDS3488.1\|NA\|GENESEQ | 3 | 3.34E−05 | NO |

### VI. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1-12. (canceled)

13. A computer-implemented method for performing a clinical genetic screening assay comprising:
obtaining a plurality of regions of interest (ROIs) comprising a set of variants, wherein the set of variants comprises pathogenic variants, likely pathogenic variants, and/or computationally predicted high impact variants;
obtaining population allele frequency information for the set of variants, wherein the population allele frequency information comprises a population allele frequency for each variant of the set of variants;
performing next generation sequencing (NGS) on a patient sample to generate NGS read data for the plurality of ROIs;
determining a read coverage for each location of each ROI of the plurality of ROIs based on the NGS read data;
obtaining a sensitivity profile, wherein the sensitivity profile provides a probability that a variant is detected based on a read coverage and a variant type of the variant;
performing a rescue minimization process comprising:
determining an expected false negative (EFN) value for each ROI of the plurality of ROIs based on the sensitivity profile and a population allele frequency of one or more variants in the ROI;
determining a sample EFN value for the patient sample based on the EFN values of the plurality of ROIs,
determining if the sample EFN value is greater than a predetermined threshold, and
when the sample EFN value is greater than the predetermined threshold,
sorting the plurality of ROIs based on the EFN values; and
rescuing a number of ROIs from the plurality of ROIs based on the sorting, wherein a sum of the EFN values of remaining ROIs of the plurality of ROIs is less than or equal to the predetermined threshold;
performing Sanger sequencing on the rescued ROIs, wherein the Sanger sequencing generates confirmatory read data for the one or more ROIs; and
outputting a result of the clinical genetic screening assay based on the NGS read data and the confirmatory read data.

14-17. (canceled)

18. The computer-implemented method of claim 13, further comprising determining the sensitivity profile by modeling variant data obtained from one or more databases based on logistic regression or using a piecewise model.

19. The computer-implemented method of claim 18, wherein when the sensitivity profile is determined using the piecewise model, and the piecewise model is a piecewise logistic regressing model.

20. The computer-implemented method of claim 19, wherein the piecewise logistic regressing model is
$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$
$$wherein :$$
$$\frac{{e}^{a + b \times T}}{1 + {e}^{a + b \times T}} = \frac{{e}^{c + d \times T}}{1 + {e}^{c + d \times T}}$$
wherein a, b, c, d, and T are predetermined parameters.

21-22. (canceled)

23. The computer-implemented method of claim 13, further comprising determining a population allele frequency for each variant of the set of variants based on variant data obtained from one or more databases comprising clinically relevant variants.

24. (canceled)

25. The computer-implemented method of claim 23, wherein a default allele frequency is determined to be the population allele frequency for a variant that is not in the one or more databases, wherein the default allele frequency is determined by extrapolating a power law using the variant data obtained from the one or more databases.

26. (canceled)

27. The computer-implemented method of claim 13, further comprising classifying variants of high confidence variants into groups comprises:
accessing sequencing read data for a benchmark variant dataset, wherein the benchmark variant dataset comprises the high confidence variants;
determining a mappability score for each of the high confidence variants;
grouping a first subset of the high confidence variants into a low mappability group, wherein each variant in the first subset has a mappability score of below a predetermined value; and
grouping remaining high confidence variants into a heterozygous single nucleotide variants group, a short heterozygous indels group, or a long heterozygous indels group based on each variant's zygosity, variant type, and variant length.

28-40. (canceled)

41. A computer-program product tangibly embodied in a non-transitory machine-readable medium, including instructions configured to cause one or more data processors to perform operations comprising:
obtaining a plurality of regions of interest (ROIs) comprising a set of variants, wherein the set of variants comprises pathogenic variants, likely pathogenic variants, and/or computationally predicted high impact variants;
obtaining population allele frequency information for the set of variants, wherein the population frequency information comprises a population allele frequency for each variant of the set of variants;
performing next generation sequencing (NGS) on a patient sample to generate NGS read data for the plurality of ROIs, wherein the patient is a subject of a clinical genetic screening assay;
determining a read coverage for each location of each ROI of the plurality of ROIs based on the NGS read data;
obtaining a sensitivity profile, wherein the sensitivity profile provides a probability that a variant is detected based on a read coverage and a variant type of the variant;
performing a rescue minimization process comprising:
determining an expected false negative (EFN) value for each ROI of the plurality of ROIs based on the sensitivity profile and a population allele frequency of one or more variants in the ROI;
determining a sample EFN value for the patient sample based on the EFN values of the plurality of ROIs,
determining if the sample EFN value is greater than a predetermined threshold, and
when the sample EFN value is greater than the predetermined threshold,
sorting the plurality of ROIs based on the EFN values; and
rescuing a number of ROIs from the plurality of ROIs based on the sorting, wherein a sum of the EFN values of remaining ROIs of the plurality of ROIs is less than or equal to the predetermined threshold;
performing Sanger sequencing on the rescued ROIs, wherein the Sanger sequencing generates confirmatory read data for the one or more ROIs; and
outputting a result of the clinical genetic screening assay based on the NGS read data and the confirmatory read data.

42-45. (canceled)

46. The computer-program product of claim 41, wherein the operations further comprise determining the sensitivity profile by modeling variant data obtained from one or more databases based on logistic regression or using a piecewise model.

47. The computer-program product of claim 46, wherein when the sensitivity profile is determined using the piecewise model, and the piecewise model is a piecewise logistic regressing model.

48. The computer-program product of claim 47, wherein the piecewise logistic regressing model is
$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$
$$wherein :$$
$$\frac{{e}^{a + b \times T}}{1 + {e}^{a + b \times T}} = \frac{{e}^{c + d \times T}}{1 + {e}^{c + d \times T}}$$
wherein a, b, c, d, and T are predetermined parameters.

49-50. (canceled)

51. The computer-program product of claim 41, wherein the operations further comprise determining a population allele frequency for each variant of the set of variants based on variant data obtained from one or more databases comprising clinically relevant variants.

52. (canceled)

53. The computer-program product of claim 51, wherein a default allele frequency is determined to be the population allele frequency for a variant that is not in the one or more databases, wherein the default allele frequency is determined by extrapolating a power law using the variant data obtained from the one or more databases.

54. (canceled)

55. The computer-program product of claim 41, wherein the operations further comprise classifying variants of high confidence variants into groups comprises:
accessing sequencing read data for a benchmark variant dataset, wherein the benchmark variant dataset comprises the high confidence variants;
determining a mappability score for each of the high confidence variants;
grouping a first subset of the high confidence variants into a low mappability group, wherein each variant in the first subset has a mappability score of below a predetermined value; and
grouping remaining high confidence variants into a heterozygous single nucleotide variants group, a short heterozygous indels group, or a long heterozygous indels group based on each variant's zygosity, variant type, and variant length.

56-68. (canceled)

69. A system comprising:
one or more data processors; and
a non-transitory computer readable medium storing instructions which, when executed on the one or more data processors, cause the one or more data processors to perform operations comprising:
obtaining a plurality of regions of interest (ROIs) comprising a set of variants, wherein the set of variants comprises pathogenic variants, likely pathogenic variants, and/or computationally predicted high impact variants;
obtaining population allele frequency information for the set of variants, wherein the population frequency information comprises a population allele frequency for each variant of the set of variants;
performing next generation sequencing (NGS) on a patient sample to generate NGS read data for the plurality of ROIs, wherein the patient is a subject of a clinical genetic screening assay;
determining a read coverage for each location of each ROI of the plurality of ROIs based on the NGS read data;
obtaining a sensitivity profile, wherein the sensitivity profile provides a probability that a variant is detected based on a read coverage and a variant type of the variant;
performing a rescue minimization process comprising:
determining an expected false negative (EFN) value for each ROI of the plurality of ROIs based on the sensitivity profile and a population allele frequency of one or more variants in the ROI;
determining a sample EFN value for the patient sample based on the EFN values of the plurality of ROIs,
determining if the sample EFN value is greater than a predetermined threshold, and
when the sample EFN value is greater than the predetermined threshold,
sorting the plurality of ROIs based on the EFN values; and
rescuing a number of ROIs from the plurality of ROIs based on the sorting, wherein a sum of the EFN values of remaining ROIs of the plurality of ROIs is less than or equal to the predetermined threshold;
performing Sanger sequencing on the rescued ROIs, wherein the Sanger sequencing generates confirmatory read data for the one or more ROIs; and
outputting a result of the clinical genetic screening assay based on the NGS read data and the confirmatory read data.

70-73. (canceled)

74. The system of claim 69, wherein the operations further comprise determining the sensitivity profile by modeling variant data obtained from one or more databases based on logistic regression or using a piecewise model.

75. The system of claim 74, wherein when the sensitivity profile is determined using the piecewise model, and the piecewise model is a piecewise logistic regressing model.

76. The system of claim 75, wherein the piecewise logistic regressing model is
$$p ( coverage ) = \{ \begin{matrix}\frac{{e}^{a + b \times coverage}}{1 + {e}^{a + b \times coverage}} , & 0 \leq coverage \leq T \\ \frac{{e}^{c + d \times coverage}}{1 + {e}^{c + d \times coverage}} , & coverage > T\end{matrix}$$
$$wherein :$$
$$\frac{{e}^{a + b \times T}}{1 + {e}^{a + b \times T}} = \frac{{e}^{c + d \times T}}{1 + {e}^{c + d \times T}}$$
wherein a, b, c, d, and T are predetermined parameters.

77-78. (canceled)

79. The system of claim 69, wherein the operations further comprise determining a population allele frequency for each variant of the set of variants based on variant data obtained from one or more databases comprising clinically relevant variants.

80. (canceled)

81. The system of claim 79, wherein a default allele frequency is determined to be the population allele frequency for a variant that is not in the one or more databases, wherein the default allele frequency is determined by extrapolating a power law using the variant data obtained from the one or more databases.

82-84. (canceled)
